(19) United States
(12) Patent Application Publication
(10) Pub. No.: US 2018/0372755 A1
(43) Pub. Date: Dec. 27, 2018

(54) Multiplexed Immunoassay for Detecting Biomarkers of Disease

(71) Applicants: Massachusetts Institute of Technology, Cambridge, MA (US); University of Massachusetts, Boston, MA (US)

(72) Inventors: Lee Gehrke, Cotuit, MA (US); Helena de Puig Guixe, Cambridge, MA (US); Irene Bosch, Brookline, MA (US); Jose Gomez-Marquez, Boston, MA (US); Kimberly Hamad-Schifferli, Arlington, MA (US)

(21) Appl. No.: 16/015,379

(22) Filed: Jun. 22, 2018

Related U.S. Application Data
(60) Provisional application No. 62/523,309, filed on Jun. 22, 2017.

(51) Int. Cl. G01N 33/68; G01N 33/569

(52) U.S. Cl. CPC G01N 33/6854; G01N 2800/26; G01N 2333/185; G01N 33/56983

(57) ABSTRACT

The present invention provides a multiplexed immunoassay which leverages stockpiled antibodies to detect whether a patient has been infected with an emerging disease which does not have specific antibodies raised against it (FIG. 1). The assay is preferably designed as a paper-based assay, which allows diagnosis at point of care (POC) and readout by eye or mobile phone. Paper-based rapid diagnostic tests (RDTs) are convenient, robust, and can be read out within minutes. The immunoassay of the invention combines the strategic use of nanoparticles of assorted colors with readily available stockpiled antibodies to one or more biomarkers of disease, particularly viral diseases.

### RELATED APPLICATION

This application claims the benefit of U.S. Provisional Application No. 62/523,309, filed on Jun. 22, 2017. The entire teachings of the above application are incorporated herein by reference.

### GOVERNMENT SUPPORT

This invention was made with Government support under grant number R33 AI100190 awarded by National Institutes of Health. The government has certain rights in the invention.

### BACKGROUND OF THE INVENTION

It is a perpetual challenge to respond to new and re-emerging infectious disease threats, which demands rapid response. Diagnostic tools are critical in the chain of response for patient care, resource allocation, disease containment, and public health surveillance. Point-of care (POC) diagnostics have gained attention for emergency situations because they are inexpensive, portable, operable by non-experts, and deliver results within minutes. For example, in lateral flow immunoassays, a biological fluid is added to the strip, and it wicks through by capillary action. Two colored lines appear for a positive test, and one line for a negative test, which can be read out by eye. The development of rapid diagnostic tools can help with providing diagnosis in the field, and has shown to be a good screening method, with several advantages over lab tests such as PCR and ELISA.

However, with every new epidemic outbreak, there is a critical initial period when biological reagents are not yet available, and an explosive rise in infections can leave clinicians with limited antibody reagents. Generating new antibodies requires an enormous amount of time and money, where the entire process from selection to manufacturing takes 16-24 months with production costs reaching hundreds of millions of dollars. The lack of a rapid antibody production method makes it difficult to serve the needs of epidemics that spread at accelerated rates, inhibiting treatment, response, and disease surveillance when it is most critical.

An opportunity lies in strategic use of cross-reactive antibodies for a closely related disease that have already been mass-produced. Seemingly, cross reactivity can be exploited to detect the biomarker of the emerging outbreak. Cross-reactivity in antibodies is common, where commercial diagnostics are often found to exhibit false positives for a similar disease. However, this lack of specificity also means that the two diseases cannot be distinguished from one another, an obvious drawback of cross-reactivity. Thus, leveraging cross-reactivity must be done in such a way that the antibodies can still distinguish the emerging virus from the original one against which it was raised. The present invention accomplishes this careful balance.

### SUMMARY OF THE INVENTION

The invention provides a multiplexed immunoassay which leverages stockpiled antibodies to detect, for example, whether a patient has been infected with an emerging disease which does not have specific antibodies raised against it (FIG. 1). The assay is preferably designed as a paper-based assay, which allows diagnosis at point of care (POC) and readout by eye or mobile phone. The invention also provides methods for adapting existing POC immunoassays, such as commercially available assays for the detection of, for example, dengue virus or zika using cross reactive antibodies to provide an immunoassay in accordance with the invention.

Paper-based rapid diagnostic tests (RDTs) are convenient, robust, and can be read out within minutes. The immunoassay of the invention combines the strategic use of nanoparticles of assorted colors with readily available stockpiled antibodies to one or more biomarkers of disease, particularly viral diseases.

Preferably, the invention provides a multiplexed immunoassay for the detection of one or more species of infectious disease biomarker protein, or another infectious disease protein that may not yet be associated with a previously identified infectious disease biomarker protein, in a biological sample, comprising the steps of: a) contacting the biological sample with one or more detection antibodies wherein at least one detection antibody of the one or more detection antibodies is cross reactive for an antigenic site on a target infectious disease biomarker protein to which the antibody was raised and is also capable of binding to an antigenic site on a different target protein, wherein at least one detection antibody of the one or more detection antibodies is capable of forming a complex with at least one target protein in the sample, wherein all of the one or more detection antibodies are labelled with a unique colorimetric label comprising a unique spectral emission; b) contacting the biological sample of step (a) with a porous matrix comprising one or more capture antibodies immobilized thereon in a capture-detection area of the porous matrix, wherein at least one capture antibody of the one or more capture antibodies is specific for an antigenic site on the target infectious disease biomarker protein to which the antibody was raised and optionally wherein at least one capture antibody of the one or more capture antibodies is capable of binding an antigenic site on a different target protein, and wherein the complexes formed in step (a) migrate through the porous matrix and contact the immobilized capture antibodies in the capture-detection area of the porous matrix; (c) detecting the differential color and intensity pattern of one or more unique spectral emissions in the capture-detection area of the porous matrix; and (d) identifying one or more species of infectious disease biomarker protein present in the sample based on the differential color and intensity pattern of the unique spectral emissions in the capture-detection area of the porous matrix. Preferably, the different target protein of step (a) is a previously unidentified infectious disease protein wherein antibodies have not previously be raised to the different target protein.

Preferably the porous matrix is a paper-based porous matrix. Preferably, the porous matrix comprises nitrocellulose. Preferably the unique colorimetric label is selected from: gold nanoparticles, colored latex beads, dye labeled beads, silver nanoparticles, quantum dots, up converting phosphors, and organic fluorophores. Preferably, the detectable label is a gold particle. Preferably, the disease biomarker is a detectable protein derived from a virus. Preferably the disease biomarker is a nonstructural protein-1 (NS1) or a glycoprotein (GP). Preferably the disease biomarker is derived from Dengue virus, Zika virus, Yellow fever virus, West Nile virus, Ebola virus, or Marburg virus.

Preferably, the invention provides a method of identifying one or more antibody pairs that are cross reactive for a target infectious disease biomarker protein and a different target protein that may not yet be associated with a previously identified infectious disease biomarker protein, comprising the steps of: (a) immobilizing each species of antibody to be tested in the capture-detection area of a porous matrix thereby providing a capture antibody; (b) labeling each species of antibody to be tested with a unique colorimetric label comprising a unique spectral emission thereby providing a detection antibody; (c) adding the labeled detection antibodies of step (b) to a biological sample comprising a predetermined amount of at least one known infectious disease biomarker protein and optionally, a predetermined amount of at least one different target protein for a sufficient time to allow the labeled detection antibodies to form a complex with one or more target proteins; (d) contacting the biological sample of step (c) with the porous matrix of step (a); (e) detecting the differential color and intensity pattern of the spectral emissions in the capture-detection area of the porous matrix; (f) identifying the antibody pairs comprising a capture antibody and a detection antibody that show specificity for only one target protein or that show cross reactivity with more than one target protein based on the differential color and intensity pattern of the spectral emissions in the capture-detection area of the porous matrix.

Preferably the invention provides a kit for the detection of zika virus, dengue virus or both in a biological sample comprising: (a) a commercially available lateral flow immunoassay for detecting dengue virus using anti-dengue antibodies specific to the NS1 protein of one or more serotypes of dengue virus; (b) an anti-dengue antibody that is cross reactive with the NS1 protein of both dengue virus and zika virus and that is labelled with a unique colorimetric label comprising a unique spectral emission thereby providing a cross-reactive detection antibody and (c) an anti-dengue antibody capture antibody known to pair with the cross reactive anti-dengue antibody of (b).

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

The patent or application file contains at least one drawing executed in color. Copies of this patent or patent application publication with color drawings will be provided by the Office upon request and payment of the necessary fee.

FIG. 1 is a schematic showing the assay of the invention and its use at the POC.

FIG. 2A is an image of solutions of the different colored nanoparticles used to provide a differential signal in the multiplexed assay (red NSs (L)) and (R) blue gold nanostars (NStar).

FIG. 2B is a TEM image of the NStar (L) and NPs (R). Scale bar=100 nm.

FIG. 2C are bar graphs showing dynamic light scattering (DLS) and zeta potential measurements of NStar (blue dashed), NStar-Ab (blue) and NS-Ab (red).

FIG. 2D is an agarose gel electrophoresis of Lane 1) red NSs conjugated to dengue 136 antibodies, Lane 2) blue NStar conjugated to dengue PAN antibodies, Lane 3) plain NStar.

FIG. 2E is a UV-vis absorption spectra of plain gold nanostars (blue dashed), NStar conjugated to antibodies (blue solid line) and red NSs conjugated to antibodies (red solid line).

FIG. 3 is a matrix that shows the results from testing of three D3V antibodies in pairs by spotting one antibody on the nitrocellulose and the other antibody on the nanoparticles, and then testing all the pairs against the four dengue serotypes, zika and a blank in human serum. Antibody 55 was conjugated on the blue GNS and the antibody 323 on the red GNP. Gray=positive tests, white=negative tests, cross-hatching=weakly positive tests.

FIG. 4A is a schematic showing GNS-55 (top) and GNP 323 (bottom).

FIG. 4B is a graph showing a titration curve of GNS-55 (squares) and GNP-323 (circles) against the antibody 411 on the nitrocellulose for D1V.

FIG. 4C is a graph showing the titration curve of GNS-55 (squares) and GNP-323 (circles) against the antibody 323 on the nitrocellulose for D1V in human serum.

FIG. 4D is a graph showing a titration curve of GNS-55 (squares) and GNP-323 (circles) against the antibody 411 on the nitrocellulose for D2V.

FIG. 4E is a graph showing the titration curve of GNS-55 (squares) and GNP-323 (circles) against the antibody 323 on the nitrocellulose for D2V in human serum.

FIG. 4F is a graph showing a titration curve of GNS-55 (squares) and GNP-323 (circles) against the antibody 411 on the nitrocellulose for D3V.

FIG. 4G is a graph showing the titration curve of GNS-55 (squares) and GNP-323 (circles) against the antibody 323 on the nitrocellulose for D3V in human serum.

FIG. 4H is a graph showing a titration curve of GNS-55 (squares) and GNP-323 (circles) against the antibody 411 on the nitrocellulose for D3V.

FIG. 4I is a graph showing the titration curve of GNS-55 (squares) and GNP-323 (circles) against the antibody 323 on the nitrocellulose for D4V in human serum.

FIG. 4J is a diagram of the detection strip assay configuration corresponding to FIGS. 4B, 4D, 4F and 4H.

FIG. 4K is a diagram of the detection strip assay configuration corresponding to FIGS. 4C, 4E 4G and 4I.

FIG. 5A is a schematic showing GNS-55 (top) and GNP 323 (bottom).

FIG. 5B is a diagram of a multiplexed detection strip assay configuration to detect the four dengue serotypes and a blank.

FIG. 5C is an image of the multiplexed assay strip showing the different signals that were exhibited for each dengue serotype.

FIG. 6A is a linear discriminant analysis (LDA) with a cross-validation of 10 plotted in its principal components and shows that it is possible to distinguish the four serotypes of dengue: D1V=triangles, D2V=X's, D3V=diamonds, D4V=squares: no Dengue=circles.

FIG. 6B is a confusion matrix showing that all the samples were accurately classified by LDA, with no off-diagonals.

FIG. 7 is a data matrix of dengue antibody pairs tested against NS1 from D1-4V and ZIKV (Z) in individual strips. Grey=a sandwich pair can form. White=no pair forms. The data shows detection of zika and dengue using only dengue antibodies.

FIG. 8A is a schematic of the nanoparticle conjugates used for detection of zika and dengue NS1.

FIG. 8B is a line graph showing individual titration tests of red GNP-136 (circles) and blue GNS-PAN, where PAN=antibody mixture of 271, 243, 626 and 411 (squares) run with immobilized 136 and D1V NS1.

FIG. 8C is a line graph showing individual titration tests of red GNP-136 (circles) and blue GNS-PAN, where PAN=antibody mixture of 271, 243, 626 and 411 (squares) run with immobilized 136 and ZIKV NS1.

FIG. 8D is a line graph showing individual titration tests of red GNP-136 (circles) and blue GNS-PAN, where PAN=antibody mixture of 271, 243, 626 and 411 (squares) run with immobilized 323 and D1V NS1.

FIG. 8E is a line graph showing individual titration tests of red GNP-136 (circles) and blue GNS-PAN, where PAN=antibody mixture of 271, 243, 626 and 411 (squares) run with immobilized 323 and ZIKV NS1.

FIG. 8F is a diagram of a detection strip assay configuration corresponding to FIGS. 8B and 8C.

FIG. 8G is a diagram of a detection strip assay configuration corresponding to FIGS. 8D and 8E.

FIG. 9A is a schematic of a schematic of a multiplexed dengue and zika assay with immobilized 2G12 at position 2, 1G11 at position 3, and control anti-Fc at position 4, and the nanoparticle conjugates used.

FIG. 9B is a diagram of a construct of a multiplexed test constructed entirely from dengue antibodies that can distinguish between NS1 from ZIKV, D1V, and a mixture.

FIG. 9C is an image of test strips when run with (L-R): NS1 from D1V and ZIKV at 1:0, 2:1, 1:1, 1:2, 0:1 where overall NS1 concentration was fixed at 1 ug/ml, which result in different colors at the two test lines depending on the amount of D1V and ZIKV NS1 present.

FIG. 10A is principal component analysis (PCA) for clustering of D1V (dark circles) ZIKV (squares) and the mixtures (grey circles), and no biomarker (grey squares).

FIG. 10B is a confusion matrix from the PCA classifiers showing correctly classified assays on the diagonal (grey), and incorrectly classified assays (off diagonals, cross-hatching).

FIG. 11A is a schematic showing the “hacking” of a commercial dengue diagnostic tool to detect zika and dengue only using dengue antibodies where blue GNS-Pan are added, and the antibody 323 is spotted down at position 2 on the nitrocellulose. The existing test line (RT-Ab) is position 3, and control line (+Ctrl) at position 4.

FIG. 11B are images of a commercial Dengue NS1 antigen test that is cross reactive.

FIG. 12A is a schematic of the nanoparticle conjugates used.

FIG. 12B is a diagram of the assay construct.

FIG. 12C is an image of the test strip results from an augmented dengue diagnostic run with L-R: DV1 NS1, DV1+zika NS1, and Zika.

FIG. 13A is a plot point graph showing PCA analysis of the resulting spots at 2 and 3 for dengue (dark gray circles), zika (medium gray circles) and the mixture (light gray circles).

FIG. 13B is a confusion matrix from PCA analysis showing correctly classified tests (grey=diagonals) and off diagonals=white.

FIG. 14 is a matrix showing screening of MARV antibody pairs with MB GP and EB GP. Hits=grey, no hits=white. Weak signal=cross-hatching. The data shows Ebola and Marburg detection using only Marburg antibodies.

FIG. 15A is a diagram of multiplexed detection with GNP-1G11 and GNS-2G12 in solution, and test strips showing immobilized 2G12 at position 2, 1G11 at position 3, and control anti-Fc at position 4.

FIG. 15B is a titration curve showing blue NStar-2G12 and red NS-1G11 run with immobilized 1G11 for EBOV-VSV.

FIG. 15C is a titration curve showing blue NStar-2G12 and red NS-1G11 run with immobilized 1G11 for MARV-VSV cell supernatants.

FIG. 15D is a titration curve showing blue NStar-2G12 and red NS-1G11 run with immobilized 2G12 for EBOV-VSV.

FIG. 15E is a titration curve showing blue NStar-2G12 and red NS-1G11 run with immobilized 2G12 for MARV-VSV cell supernatants.

FIG. 15F is a schematic of the conjugation particles used.

FIG. 16A is a schematic of the conjugated nanoparticles used.

FIG. 16B is a schematic of multiplexed detection with GNP-1G11 and GNS-2G12 in solution, and test strips showing immobilized 2G12 at position 2, 1G11 at position 3, and control anti-Fc at position 4.

FIG. 16C is an image of test strips run with NStar-2G12 and NS-1G11 and MARV-GP and EBOV-GP at varying MARV-GP:EBOV-GP ratios.

FIG. 17A shows PCA analysis from six to two dimensions and showing Ebola (black squares) Marburg (black circles), mixtures of Ebola and Marburg (gray circles), and no GP,ø (gray circles).

FIG. 17B shows the confusion matrix with 100% classification accuracy, with correct predictions (diagonals=grey) and no off-diagonals (white).

FIG. 18 is a table showing the values of limits of detection (LOD) and K_{DS} obtained from Langmuir fits for dengue 3 antibodies when binding to dengue NS1 of serotypes 1-4. NC=antibody immobilized on the nitrocellulose.

FIG. 19 is a confusion matrix for mixtures of dengue serotypes when detected with dengue 3 antibodies, reporting a 81% classification accuracy.

FIG. 20 is a pie chart showing the cross reactivity of dengue 3 antibodies for other dengue serotypes.

FIG. 21A is a table showing sequence identity between Zika and Dengue 1-4 NS1.

FIG. 21B is a table showing sequence identity between Ebola and Marburg GP.

FIG. 22 is a table showing complete pairs of dengue and zika antibodies for zika and dengue 1-4 NS1. Strong binding pairs=gray. Non-binding pairs=white, Weak binding pairs=cross-hatching.

FIG. 23 is a schematic showing peptide mapping of antibodies (mAb323, mAb411 and mAb 243, mAb 626, mAb 271 and mAb 136) generated against dengue NS1 show a range of cross-reactivities with different dengue serotypes.

FIG. 24 is a schematic showing peptide mapping of antibodies (mAb323, mAb411 and mAb 243, mAb 626, mAb 271 and mAb 136) generated against dengue NS1 show a range of cross-reactivities with different dengue serotypes and zika NS1.

FIG. 25A is a schematic of strip and nanoparticle-antibody conjugates run. FIG. 25B is a line graph showing individual titration tests with immobilized 136 run with red nanospheres conjugated to 136 (circles) and blue nanostars conjugated to the PAN mixture of antibodies (squares) run with DV1 NS1.

FIG. 25C is a line graph showing individual titration tests with immobilized 136 run with red nanospheres conjugated to 136 (circles) and blue nanostars conjugated to the PAN mixture of antibodies (squares) run with DV2 NS1.

FIG. 25D is a line graph showing individual titration tests with immobilized 136 run with red nanospheres conjugated to 136 (circles) and blue nanostars conjugated to the PAN mixture of antibodies (squares) run with DV3 NS1.

FIG. 25E is a line graph showing individual titration tests with immobilized 136 run with red nanospheres conjugated to 136 (circles) and blue nanostars conjugated to the PAN mixture of antibodies (squares) run with DV4 NS1.

FIG. 25F is a line graph showing individual titration tests with immobilized 136 run with red nanospheres conjugated to 136 (circles) and blue nanostars conjugated to the PAN mixture of antibodies (squares) run with Zika NS1.

FIG. 25G is a line graph showing individual titration tests with immobilized 323 run with red nanospheres conjugated to 136 (circles) and blue nanostars conjugated to the PAN mixture of antibodies (squares) run with DV1 NS1.

FIG. 25H is a line graph showing individual titration tests with immobilized 323 run with red nanospheres conjugated to 136 (circles) and blue nanostars conjugated to the PAN mixture of antibodies (squares) run with DV2 NS1.

FIG. 25I is a line graph showing individual titration tests with immobilized 323 run with red nanospheres conjugated to 136 (circles) and blue nanostars conjugated to the PAN mixture of antibodies (squares) run with DV3 NS1.

FIG. 25J is a line graph showing individual titration tests with immobilized 323 run with red nanospheres conjugated to 136 (circles) and blue nanostars conjugated to the PAN mixture of antibodies (squares) run with DV4 NS1.

FIG. 25K is a line graph showing individual titration tests with immobilized 323 run with red nanospheres conjugated to 136 (circles) and blue nanostars conjugated to the PAN mixture of antibodies (squares) run with zika NS1.

FIG. 25L is a diagram of multiplexed detection with NS mAb 136 at position 3, NS mAb 323 at position 2 and blank at position 1.

FIG. 25M is a diagram of multiplexed detection with NS mAb 136 at position 3, NS mAb 323 at position 2 and control anti Fc at position 4.

FIG. 26A is a point graph of Dengue 1-4 NS1 and zika NS1 mixtures.

FIG. 26B is a confusion matrix and PCA. Gray=diagonals, correct predictions. White=off diagonals, no incorrect predictions. Cross-hatching=off-diagonals, incorrect predictions.

FIG. 27 is a matrix showing LODs and K_{DS} of pairs with Dengue and zika NS1 for antibodies on the nanoparticles (NP) and nitrocellulose (NC).

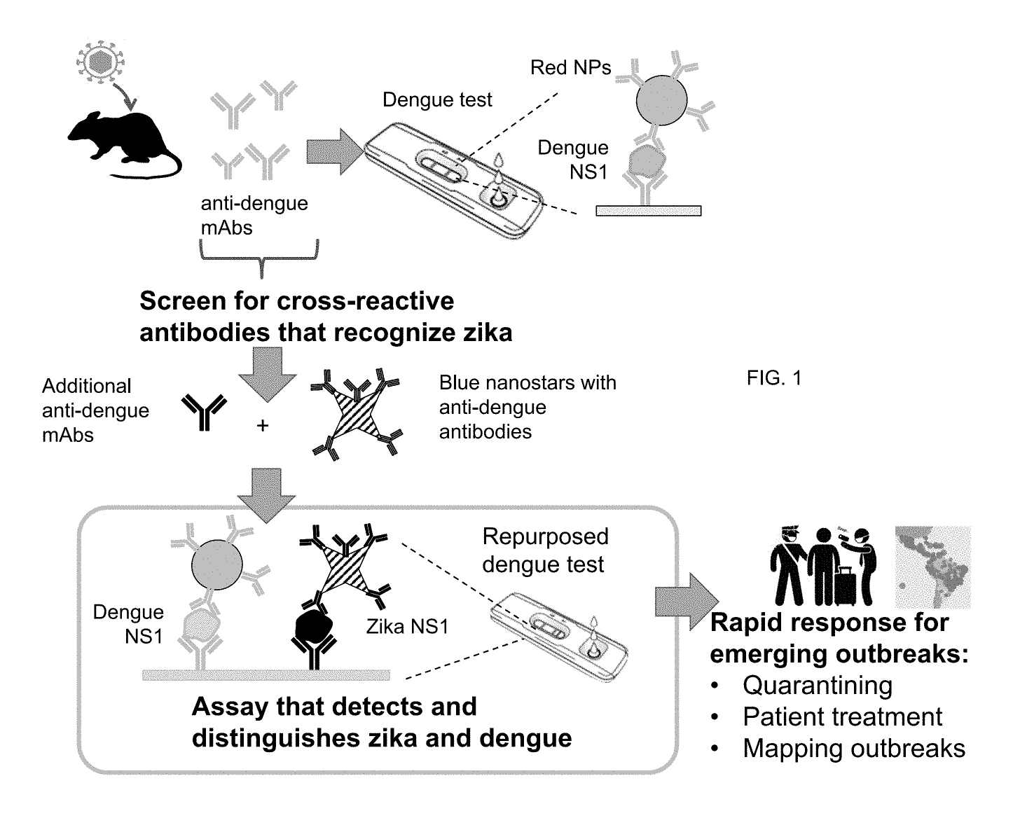

### DETAILED DESCRIPTION OF THE INVENTION

The terms “a”, “an” and “the” as used herein are defined to mean “one or more” and include the plural unless the context is inappropriate.

As is known in the art, an “antibody” is an immunoglobulin that binds specifically to a particular antigen. The term encompasses immunoglobulins that are naturally produced in that they are generated by an organism reacting to the antigen, and also those that are synthetically produced or engineered. An antibody may be monoclonal or polyclonal. An antibody may be a member of any immunoglobulin class, including any of the human classes: IgG, IgM, IgA, and IgD.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. However, some monoclonal antibodies may cross react with similar antigenic sites on different proteins.

The term “cross reactive antibody” or “cross reactive antibodies” also referred to herein as “non-unique antibody or antibodies” refers to one or more antibodies that are capable of binding to similar antigenic sites on multiple target proteins either as a single binding antibody or as paired with a second antibody (i.e. an “antibody pair”).

The term “PAN” refers to a combination of antibodies or a single antibody that can bind all the types of flaviviruses, PAN dengue, would be antibodies binding dengue 1, 2, 3, 4 NS1. PAN Flavi, would bind all different flaviviruses, etc.

The terms “polypeptide”, “peptide”, and “protein”, as used herein, are interchangeably and are defined to mean a biomolecule composed of amino acids linked by a peptide bond.

The term “subtype” or “serotype” is used herein interchangeably herein refers to genetic variants of, for example, a virus antigen such that one subtype is recognized by an immune system apart from a different subtype. For example, dengue virus subtype 1 (DV1) is immunologically distinguishable from dengue virus subtype 2 (DV2).

As used in this invention, the term “epitope” means any antigenic site on an antigen to which the antibody binds.

The word “complex” as used herein refers to the product of a specific binding agent-ligand reaction. Preferably, the term “complex” as used herein refers to a labelled detection antibody bound to its target biomarker prior to being detected by and bound to a capture antibody in an immunoassay.

The term “antigen” and/or “analyte” refers to a polypeptide or protein that is able to specifically bind to an antibody and form a complex. The site on the antigen with which the antibody binds is referred to as an antigenic determinant or epitope.

The term “infectious disease proteins”, “infectious disease biomarker protein” or “biomarker” as used herein refers to protein or peptides molecules that can be measured in the patient that provide early detection of an emerging or established infectious disease in the patient. Many previously identified infectious disease biomarkers associated with various infectious diseases are known the those skilled in the art and may also be found for example in the Infectious Disease Biomarker Database (IDBD).

The invention provides methods of targeting proteins in biological samples associated with infectious diseases wherein the proteins are not previously identified infectious disease biomarker proteins and/or wherein there are no known or readily available antibodies raised specifically to that target protein. These proteins may be referred to herein as “different target protein(s)”.

“Lateral flow assays (LFA)” as that term is used herein are immunoassays that can be used to detect infectious disease biomarkers in a biological sample. The general format of LFA uses rationale similar to that of an ELISA. Lateral flow technology is well-suited to point-of-care (POC) disease diagnostics because it is robust and inexpensive, without requiring power, a cold chain for storage and transport, or specialized reagents. Many LFA devices comprise a porous matrix capable of supporting the test and which is made of a material which can absorb a liquid sample and which promotes capillary action of liquid sample along the porous matrix, such as nitrocellulose. The porous matrix may come in any shape or size, one common size being a strip that is capable of being held in a hand. In one exemplary LFA test format, sometimes referred to as “dipstick” or “half-strip”, the labelled antibodies and biological sample are present in a container such as a test tube, wherein they become conjugated and form a complex. A nitrocellulose membrane, for example, with a capture antibody bound to it at a capture-detection area is contacted with the labelled complex of detection antibody and target infectious disease biomarker in the container and migrates toward the capture-detection area where it is captured by the capture antibody, becomes immobilized and produces a distinct signal, for example a colored line. Some lateral flow assays may have more than one test line for multiplex testing for multiple infectious disease biomarkers and are one example of a “multiplexed immunoassay”. As used herein, the term “lateral flow” refers to capillary flow through a material in a horizontal direction but will be understood to apply to the flow of a liquid from a point of application of the liquid to another lateral position even if, for example, the device is vertical or on an incline. Lateral flow depends upon properties of the liquid/substrate interaction (surface wetting or wicking action) and does not require or involve application of outside forces, e.g., vacuum or pressure applications by the user.

As used herein, the term “porous matrix” or “porous material” refers to a material capable of providing capillary movement or lateral flow. This would include material such as nitrocellulose, nitrocellulose blends with polyester or cellulose, untreated paper, porous paper, rayon, glass fiber, acrylonitrile copolymer or nylon or other porous materials that allow lateral flow. Porous materials useful in the immunoassays described herein permit transit, either through the porous matrix or over the surface of the material, of particle label used in these devices.

By “capillary flow”, it is meant liquid flow in which all of the dissolved or dispersed components of the liquid are carried at substantially equal rates and with relatively unimpaired flow laterally through the membrane, as opposed to preferential retention of one or more components as would occur, e.g., in materials capable of adsorbing or imbibing one or more components.

As used herein, the term “specifically binds” refers to the specificity an antibody used in accordance with the invention such that the antibody primarily binds to a defined virus protein and does not generally bind to similar family member corresponding same protein. An antibody “specifically binds” to an infectious disease biomarker, for example, if it binds with unique or greater affinity, avidity, more readily, and/or with greater duration than it binds to other substances. Recognition by an antibody of a target biomarker in the presence of other potential interfering substances is also one characteristic of specifically binding. Preferably, “cross-reactive” antibodies used in the methods of the invention bind to one or more closely related antigenic sites on one or more target infectious disease biomarkers.

As used herein, the term “ colorimetric label” includes, but is not limited to colored latex (polystyrene) particles, colored polymeric particles, colored cellulose particles, metallic (e.g., gold) sols including gold nanoparticles, non-metallic elemental (e.g., Selenium, carbon) sols and dye sols.

Preferred colorimetric labels of the invention include “gold nanoparticles”, which may be designated interchangeably herein as “gold nanostars”, “nanostars”, “nanoparticles”, “GNS”, “GNP”, “NP”, “NS” or “NStar”.

The term “biological sample,” as used herein, refers to a sample of biological origin, or a sample derived from the sample of biological origin, preferably from human patient. The biological samples include, but are not limited to, blood, plasma, serum, saliva, cerebral spinal fluid, pleural fluid, milk, lymph, sputum, semen, urine, stool, tear, saliva, needle aspirate, external section of the skin, respiratory, intestinal, or genitourinary tract, tumor, organ, cell culture, cell culture constituent, tissue sample, tissue section, whole cell, cell constituent, cytospin, or cell smear. The term “biological sample” does not include samples containing target protein biomarkers that have been denatured or otherwise altered such that the protein is no longer in its native configuration.

The terms “patient” of “subject” as used herein refers to an animal. Preferably the animal is a mammal. More preferably the mammal is a human. A “patient” also refers to, for example, dogs, cats, horses, cows, pigs, guinea pigs, fish, birds and the like.

As used herein, the terms “sequence identity” or “sequence similarity” refers to the overall relatedness between polymeric molecules, e.g., between nucleic acid molecules (e.g., DNA molecules and/or RNA molecules) and/or between polypeptide molecules. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

As used herein, the term “substantially” refers to the qualitative condition of exhibiting total or near-total extent or degree of a characteristic or property of interest.

The phrase “substantial identity” or “substantially identical” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, Preferably, two sequences are considered to be substantially identical if at least 80%, 85%, 90%, 91%, 92%, 93%, 94%, 95%, 96%, 97%, 98%, 99% or more of their corresponding residues are identical over a relevant stretch of residues. Preferably, the relevant stretch is a complete sequence but could be less than the complete sequence.

### DESCRIPTION OF INVENTION

The multiplexed immunoassay of the invention leverages stockpiled antibodies for rapid and early detection of an emerging infectious disease outbreak for example, a viral outbreak such as zika or ebola. Traditional paper-based immunoassays rely on antibodies raised specifically to an antigen of interest, and only one color for a test line (typically red).

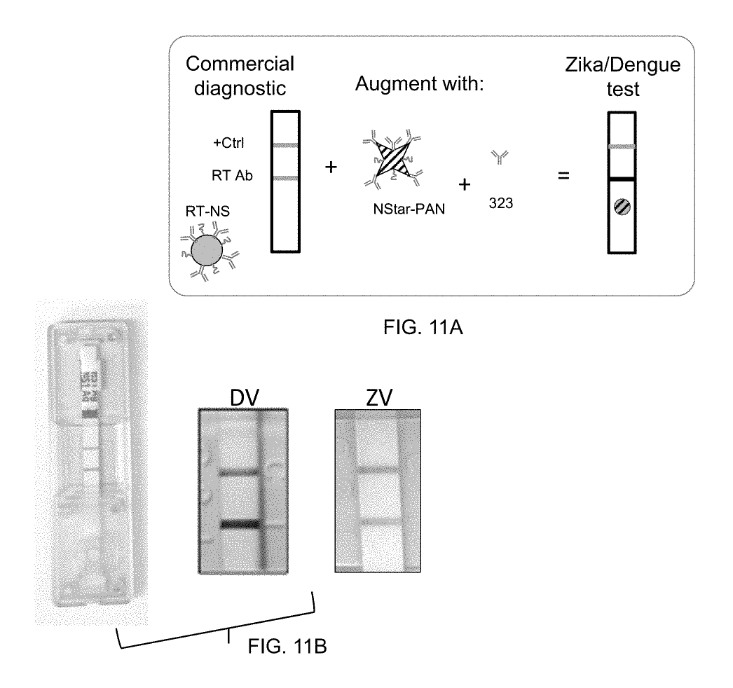

The invention also provides methods for adapting existing POC immunoassays, such as commercially available assays for the detection of, for example, dengue virus or zika using cross reactive antibodies to provide an immunoassay in accordance with the invention. As described in Example 1 and shown in FIG. 11, a commercial dengue diagnostic can be modified or “hacked” to detect zika virus and dengue virus using only dengue antibodies. For example, a cross reactive dengue antibody labelled with a colorimetric detection label is added to the dengue specific antibodies present in the commercially available diagnostic. An additional dengue capture antibody known to pair with the cross-reactive detection antibodies is added to the commercial immunoassay thereby transforming the commercially available dengue virus immunoassay into an assay capable of detecting dengue and/or zika in a biological sample.

The present invention combines the use of cross-reactive antibodies for a closely related infectious disease antigen in combination with multicolored nanoparticles to detect an emerging infectious disease antigen while still distinguishing it from the antigen for which the antibodies were raised. This approach has not been demonstrated before in that it leverages cross-reactivity of antibodies, which is typically viewed as a drawback.

Compared to existing immunoassays, the multiplexed immunoassay of the invention can be deployed within weeks of a new outbreak, as opposed to 1 year. For example, the Corgenix Medical Corp. (CO USA) rapid test for Ebola Virus took 11 months to develop and deploy, and the Biocan Diagnostics Inc. (Canada) zika test took 10 months, even with accelerated regulatory mechanisms.

As discussed in Example 1, we have discovered that antibodies for non-structural protein 1 (NS1) from dengue serotype 3 may be used to detect and distinguish NS1 from dengue and zika. We screened dengue antibodies and selected a subset of these that can bind to each of the serotypes. Two of these antibodies were linked to gold nanoparticles of two different colors, and two are immobilized on the paper as the other half of the test. Then, when a biological sample comprising dengue or zika is run in the assay, a different colored pattern results depending on the biomarker present. For readout, we developed image analysis to enable diagnosis simply by distinguishing the color of the assay. In addition, we were able to demonstrate the same principle of repurposing antibodies to detect all 4 serotypes of dengue using only dengue 3 (DV3) antibodies. Additionally, we demonstrated that it could be applied to detect Ebola virus biomarker glycoprotein 1 (GP1) using Marburg anti-GP antibodies.

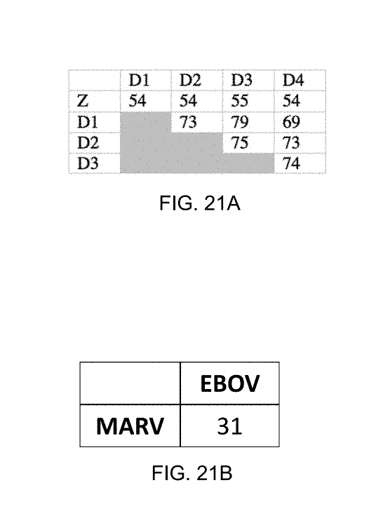

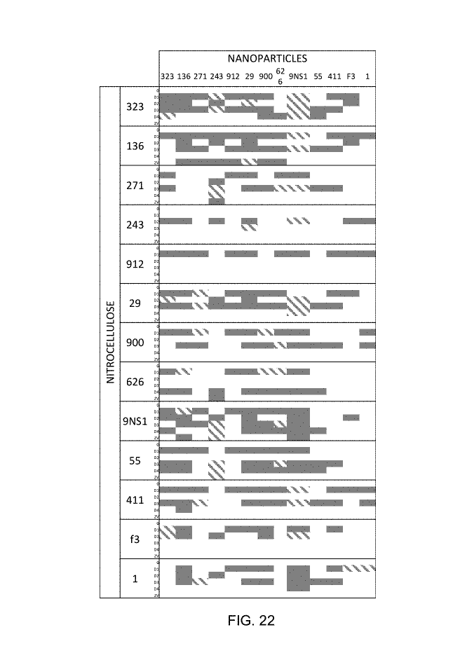

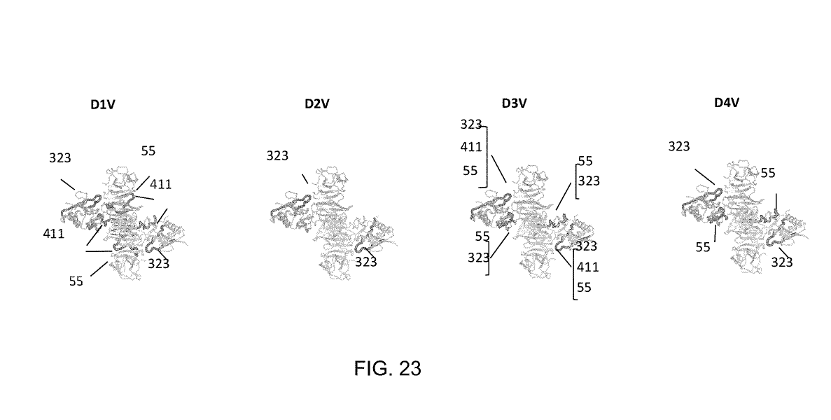

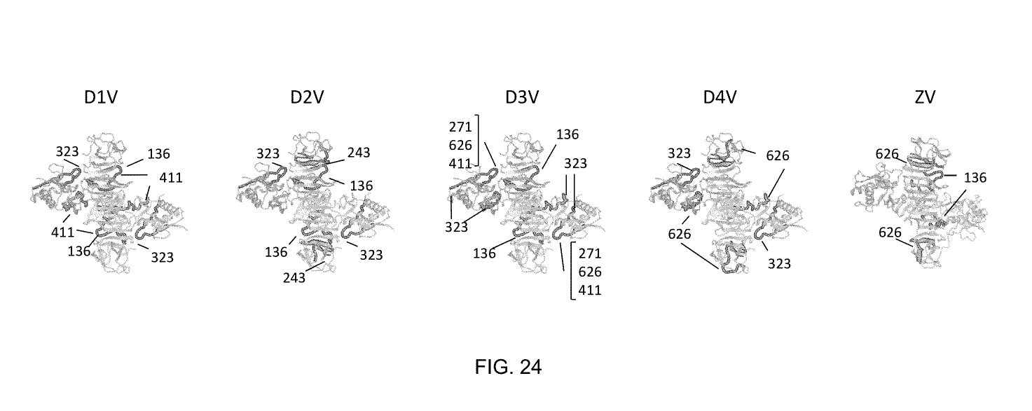

The cross-reactive antibodies used in in the immunoassay of the invention may be any known antibody capable of binding an infectious biomarker associated with an infectious disease. Preferably the biomarker is a detectable protein biomarker derived from a viral infectious disease. Preferably, the higher the sequence similarity between biomarkers facilitates exploiting cross reactivity. In general, there is higher biomarker sequence similarity between corresponding and highly conserved proteins within the same virus family. For example, NS1 serves as a convenient target biomarker for detecting and diagnosing infection of a human patient with one or more viruses from the flavivirus family such as serotypes of dengue virus, zika virus, Yellow Fever virus, Powassan, and a host of other viruses. Viral glycoproteins (GP) also serve as convenient target biomarker for detecting and diagnosing infection of a human patient with one or more viruses from the filovirus family such as Ebola, Marburg, and others, and the alphavirus family such as Chikungunya, and others. Also, envelope proteins (E proteins) from any virus could serve as potential targets. Sequence identity between zika and dengue serotypes 1-4 NS1 and between Ebola and Marburg GP is shown in FIG. 21A and FIG. 21B.

This strategy is applicable to viruses of other families. For example, in addition to flaviviruses and filoviruses, this includes but is not limited to, arenaviruses, bunya viruses, paramyxoviruses, alphaviruses, herpes viruses.

To detect biomarkers of a virus using antibodies for biomarkers from another virus, the antibodies should include one cross-reactive species, i.e., that can bind to both the virus it was raised against and also the new antigen of interest. In addition, it should include antibodies that are specific to the antigen against which it was raised, or the old antigen. The cross-reactive antibody should be linked to the nanoparticles of one color, and then the more specific antibodies should be linked to the nanoparticles of the other color. Also, for the immobilized antibodies, the cross-reactive vs. specific antibodies should be spatially separated on the paper. In doing so, the signal that results for when the new antigen vs. the old one is distinguishable.

Preferably, the detection and capture antibodies used in the immunoassay of the invention are monoclonal antibodies that are readily available for use in an assay as soon as a potential infectious disease outbreak is identified. These antibodies are referred to herein as “stockpiled” antibodies to one or more infectious diseases may be obtained, for example from academic and industry laboratories, such as the Strategic National Stockpile from the Center for Disease Control (CDC) and the Department of Health and Human Services (DHHS), FDA, ATCC, NIH, WHO. Stockpiled antibodies may also be identified in the scientific and patent literature. For example, antibodies 1, 55, 243, 271, 323, 411, 626, 912 used in the experiments of Example 1 herein are described in International Publication Number: WO 2017/139587.

There are a variety of paper-based assay formats known to those of ordinary skill in the art for using antibodies to detect an infectious disease biomarker in a sample which can be effectively employed in the assay of the invention. Preferably, the assay is an enzyme linked immunosorbent assay (ELISA)-sandwich assay, preferably in a lateral flow format.

Preferred lateral flow format assays are described in United States Patent Application Publication 2017/0234866. In accordance with the invention, antibodies that that are specific to one or more related antigens of one or more infections disease markers are conjugated to a colorimetric label such as a gold nanoparticle or nanostar and thereby provides a “detection antibody”. The detection antibody is contacted with the biological sample that may contain one or more infectious disease biomarkers for a period sufficient to allow for the formation of a complex between the detection antibody and the infectious disease biomarker. The biological sample comprising any potential complex is then contacted with the porous, preferably paper-based matrix. The biological sample migrates along the membrane to the capture-detection area where a second antibody, referred to herein as the “capture antibody” is immobilized and binds to a specific infections disease biomarker or other, possibly previously unidentified, protein target thereby forming a sandwich of the detection antibody, antigen and capture antibody. The color and intensity pattern of the colorimetric label at the capture-detection area indicates the presence of one or more infectious disease biomarkers in the sample. Such tests can typically be performed with a very small amount of biological sample.

The method for determining cross-reactive antibody pairs is to immobilize each of the antibodies onto a nitrocellulose or paper strip. Also, each of the antibodies are conjugated to a colorimetric label such as gold nanoparticle. Then, each nitrocellulose strip is run as a dipstick immunoassay for the antigen with each of the antibodies. This involves immersing the strip into a solution containing the antigen and also the nanoparticle-antibody conjugate. The fluid wicks up and results in a colored spot at the area of the immobilized antibody if both the immobilized antibody and the nanoparticle-antibody conjugate bind to the antigen simultaneously. This is repeated for all of the relevant antigens, which must include the antigen against which the antibodies were raised, as well as the new antigen of interest.

Each species of unique cross-reactive detection antibody is preferably labeled with a unique colorimetric label comprising a unique spectral emission. Suitable colorimetric labels include gold nanoparticles, colored latex beads, magnetic particles, carbon nanoparticles, selenium nanoparticles, silver nanoparticles, quantum dots, up converting phosphors, organic fluorophores, textile dyes, enzymes, liposomes.

Any detectable label recognized in the art as being useful in various assays could be used in the present invention. Preferably, the detectable label having a unique spectral emission includes, but is not limited to, noble metal nanoparticles (NP) such as gold or silver nanoparticles, colored latex beads, quantum dots, up converting phosphors, organic fluorophores and enzymes. Preferably the detectable labels provide a direct spectral signal at the completion of the assay such as the color detectable color from metal nanoparticles. Color/fluorescence release from an enzyme conversion for example requires an extra step to produce a spectral signature which is preferably avoided.

### EXAMPLES

The present invention will be better understood in connection with the following Examples. However, these examples are for illustrative purposes only and are not meant to limit the scope of the invention. Various changes and modifications will be apparent to those skilled in the art and such changes and modifications including, without limitation, those relating to the formulations and/or methods of the invention may be made without departing from the spirit of the invention and the scope of the appended claims.

### Example 1

### Immunoassays Based on Cross-Reactive Antibodies

### Abstract

We present a multiplexed immunoassay (IA) by exploiting the use of both specific and cross-reactive antibodies with gold nanoparticles of different colors. This allows us to provide a specific diagnostic for two related diseases, such as DENV and ZIKV; or Marburg and Ebola by using antibodies raised against only one of the two related diseases. Because the biomarkers are related, cross reactivity can be observed, but using different colored NPs can ameliorate to enable distinguishing between single or mixed infections.

### Introduction

The frequency of new outbreaks are rising globally¹, and it is a perpetual challenge to respond to new and re-emerging disease threats. Diagnostic tools are critical in the chain of rapid response for patient care, resource allocation, disease containment, and public health surveillance. In particular, point-of care (POC) diagnostics have gained attention for emergency situations because they are inexpensive, portable, operable by non-experts, and deliver results within minutes.² In POC immunoassays, a biological fluid wicks through a strip by capillary action. Two lines appear for a positive test, and one line for negative, which can be read by eye. Rapid diagnostic tools have several advantages over PCR and ELISA, and can aid diagnosis in the field².

With every new epidemic, there is an explosive rise in infections at the initial stages of the outbreak. Because this is when biological reagents are not yet available, clinicians are left with limited options during a period when disease containment is most critical. Antibodies are a vital biological reagent, but selecting and manufacturing new ones can take 16-24 months, with production costs reaching $100M.³⁻⁴ Clearly, the lack of a rapid antibody production method impedes confinement epidemics that spread at accelerated rates,⁵ inhibiting treatment, response, and disease surveillance when it is most needed.

An opportunity lies in strategic use of cross-reactive antibodies for a closely related disease that have already been mass-produced. Antibody cross-reactivity is common, where commercial diagnostics often exhibit false positives for similar diseases.⁶

Seemingly, it can be exploited to detect the biomarker of an emerging outbreak. However, this lack of specificity means that diseases cannot be distinguished from one another, which can result in dangerous outcomes for co-circulating pathogens.⁷ This is particularly problematic for the zika, dengue, and chikungunya outbreaks, as they share the mosquito vector Aedes Aegypti, have similar non-specific initial symptoms, but have drastically different outcomes. ⁸,⁹⁻¹⁰ Thus, leveraging cross-reactivity must be done in such a way that the emerging virus can be distinguished from the original one against which it was raised.

A solution is to exploit the properties of the nanoparticles (NPs) responsible for the test line signal. Gold NPs absorb strongly in the visible and their color can be tuned by changing their size, producing different test line colors.¹¹ Here, we exploit antibody cross-reactivity and NP properties to enable differential specificity of antibodies.

We demonstrate that with only antibodies for dengue virus serotype 3 (DV3), a multiplexed assay that can distinguish all four dengue serotypes can be constructed without specific antibodies raised against each serotype. We also construct a multiplexed zika and dengue assay entirely of dengue antibodies.

We extend the strategy to filoviruses to construct a multiplexed Ebola and Marburg glycoprotein (GP) assay using only Marburg antibodies. The approach can be used to augment a commercial dengue antigen test so that it can detect both dengue and zika.

### Results

In an immunochromatography assay, NP-antibody (NP-Ab) conjugates are mixed with a patient sample and flowed through a porous nitrocellulose membrane, which has antibodies specific for the biomarker immobilized on the test area. If antigen is present, it binds to the NP-Ab, and the complex migrates though the nitrocellulose and accumulates at the test line via binding of the antigen to the immobilized antibodies, resulting in a dot visible due to the NP. The control area has antibodies that can bind to the antibodies on the NP, so signal here indicates proper sample migration. Therefore, a positive test results in NP accumulation at both the control and test bands.

Distinguishing All Serotypes of Dengue with D3V Antibodies

First, we investigated whether antibodies specific for dengue serotype 3 (DV3) could be used to detect and distinguish dengue serotypes 1-4. The dengue biomarker is nonstructural protein 1 (NS1), which is secreted during infection.¹² NS1 varies among the serotypes and has a sequence similarity of ˜75%, so cross reactivity between antibodies is high.

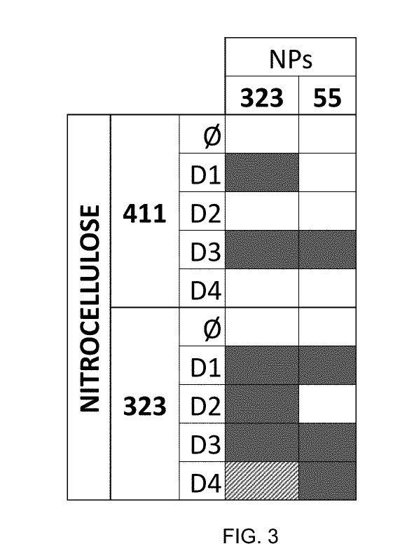

Antibodies against D3V NS1 were generated (Methods) and then screened for their ability to bind in pairs to NS1 from D1V, D2V, D3V, and D4V. For screening, each D3V antibody was conjugated to purchased gold NPs (GNPs, Innova) and also immobilized onto nitrocellulose at the test line (Methods). Strips were run with NS1 of each dengue serotype, and combinations of antibodies that resulted in a positive (FIG. 3, gray), negative (white), and weakly positive tests (cross-hatching) were recorded.

As expected, all pairs recognized D3V NS1. Pairs which exhibited crossover reactivity were identified. For example, antibody 323 conjugated to the NPs and 323 immobilized on the paper (denoted NP-323/paper-323) could bind to NS1 from all 4 serotypes. More importantly, some antibodies were cross reactive to only certain serotypes, such as antibody 411 showing cross-reactivity with D1V, but not D2V or D4V. This partial cross reactivity was key in determining a configuration of antibody pairs that could distinguish between the serotypes.

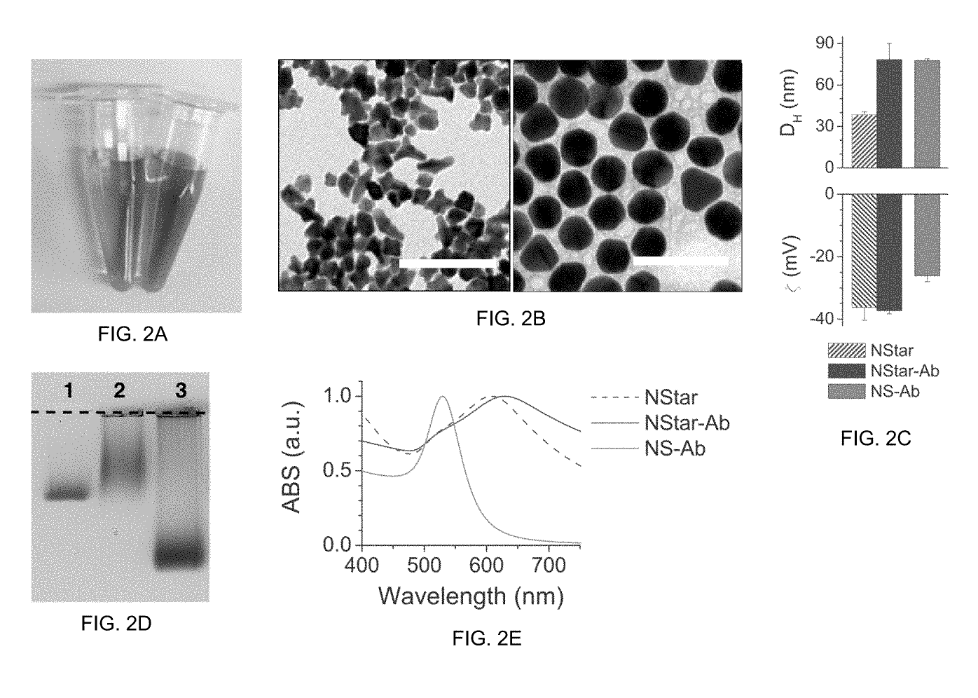

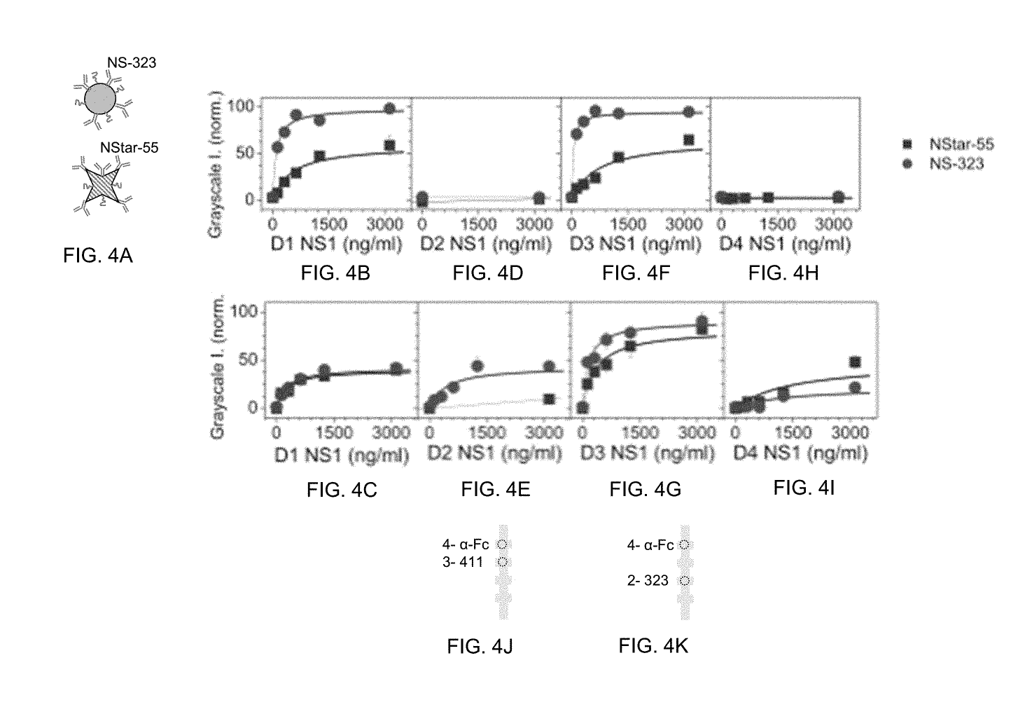

Using the screening matrix, we chose an assay configuration that could provide a differential colorimetric signal depending on the serotype present. This required NPs of different colors, so 30 nm blue gold nanostars (GNS) were synthesized ¹³⁻¹⁴ and 40 nm diameter red GNPs were purchased (Innova Biosciences). Particles were characterized by TEM, optical absorption, and dynamic light scattering (FIGS. 2A-2E). The configuration used antibodies 323 and 411 spotted on areas 2 and 3, respectively, and red GNPs were conjugated to antibody 323, blue GNSs to antibody 55. Strips included a positive control location and a blank location for signal normalization (FIGS. 4A-4K).

First, we quantified the resulting RGB intensity for each of the immobilized antibodies in response to each serotype (FIGS. 4B-4I. NS1 of the four dengue serotypes was spiked into human serum at varying concentrations and run with either immobilized 323 or 411. Immobilized 411 run with GNP-323 (circles, FIGS. 4B, 4D, 4F and 4H, respectively and GNS-55 (squares, FIGS. 4B, 4D, 4F and 4H, respectively, showed test line intensities that increased and then saturated with increasing D3V NS1 (FIG. 4F). This suggests immobilized 411 formed a high affinity sandwich for D3V NS1 and the GNP-323 and GNS-55, which was expected as the antibodies were raised against D3V NS1. Moreover, for no NS1, neither antibody pair showed signal, confirming that test signal was due to NS1 presence and not nonspecific binding. Similar curves were observed for immobilized 323 (FIG. 4G).

Importantly, test line intensities differed between the blue GNS-55 and red GNP-323. This can be attributed to differences in Ab coverage on the NPs, strength of the NP optical absorption, and the differing binding affinities of 55 vs. 323 for D3V NS1. These intensity differences are key for differential signal in the multiplexed test. We calculated limits of detection (LODs)¹⁵ and dissociation constants (K_{D}) of the pairs for NS1 from all four dengue serotypes by fitting to a modified Langmuir equation.¹⁶⁻¹⁷ LODs were 4-400 ng/ml, below NS1 concentrations found in dengue infected patients.^{12, 18}

D1V NS1 exhibited similar curves with a slightly lower intensity, which was expected given that D1V-NS1 and D3V-NS1 have the highest sequence identity among the serotypes (79%, FIG. 21A). Neither NS1 from D2V nor D4V were able to bind to the immobilized Ab 411 (FIGS. 4D and 4H), which was expected from the pairwise analysis (FIG. 3).

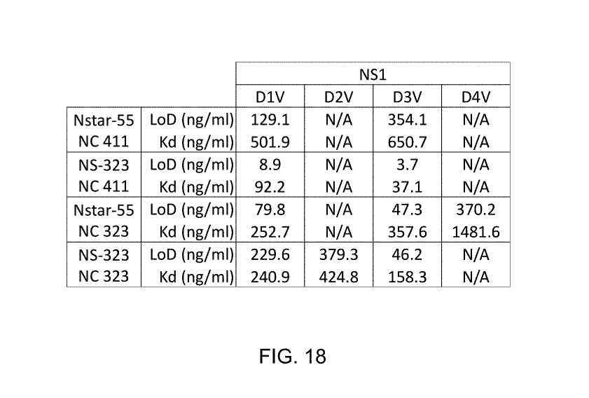

Immobilized Ab-323 run with red GNP-323 and blue GNS-55 (red circles and blue squares, FIGS. 4C, 4E, 4G and 4I, respectively) also exhibited intensity curves that varied with serotype, and intensities differed from those for immobilized Ab-411. Both NPs could form sandwiches with 323 for D3V NS1 (FIG. 4G), where red GNP-323 exhibited a slightly stronger signal at high concentrations than the blue GNS-55. Again, cross-reactivity was observed with D1V NS1 with intensities similar for GNP-323 and GNS-55 (FIG. 4C). D2V NS1 was detectable with GNP-323, but with GNS-55 showed negligible signal (FIG. 4E). D4V was detectable by GNS-411, but less so for GNP-323 (FIG. 4I). LODs and K_{D} values were calculated from Langmuir fits and were in similar ranges for immobilized Ab 411 (FIG. 18).

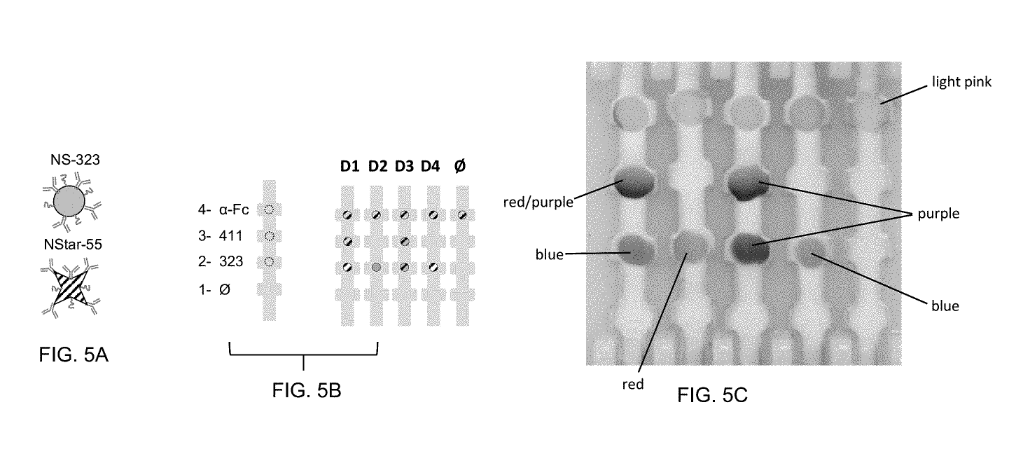

Using this information, we tested the multiplexed detection using a strip with separate areas for antibody 323, 411, and anti-Fc (FIGS. 5A-5C) run with a mixture of GNP-323+GNS-55 in serum. Different signals were exhibited for each of the serotypes. A purple dot appeared as positions 2 and 3 for strips run with D3V NS1, indicating D3V NS1 could bind to both 323 and 411. When D1V was present, a blue dot formed at area 2 and a red/purple dot at 3, indicating sandwich formation of GNS-55 with 323 and both GNP-323 and GNS-55 with 411 (FIG. 5C). When D2V NS1 was present, test area 2 showed a red spot, area 3 did not (FIG. 5C), suggesting that GNP-323 and GNS-411 did not form a sandwich with immobilized 411. When D4V NS1 was present, a blue dot appeared at area 2 but no dot appeared at area 3, indicating that GNS-55 formed a sandwich with immobilized 323 but not immobilized 411 (FIG. 5C). For serum with no NS1, only the positive control area 4 showed a spot, confirming a negative test (FIG. 5C).

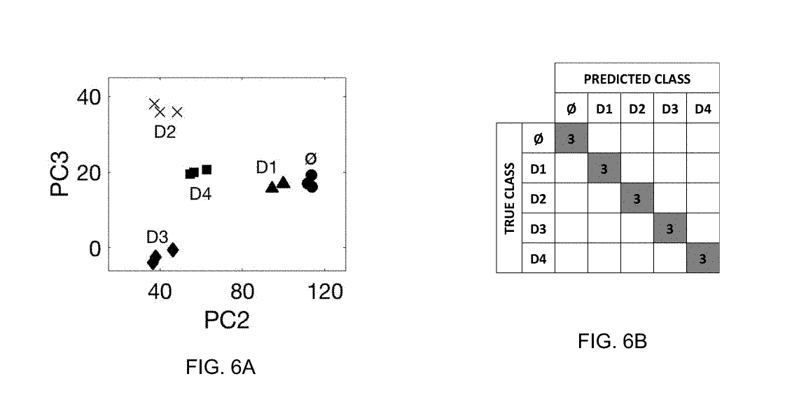

Clearly, the resulting test line pattern and color varies depending on the serotype present. RGB values of test areas were quantified¹⁹ for each serotype. We trained a linear discriminant analysis (LDA) with six predictor variables (RGB values of spots 2 and 3) to distinguish the four dengue serotypes. A confusion matrix was used to plot LDA results, where diagonal values show correctly classified responses, and off-diagonals misclassified responses. LDA was able to successfully separate the four serotypes and blank (FIG. 6B).

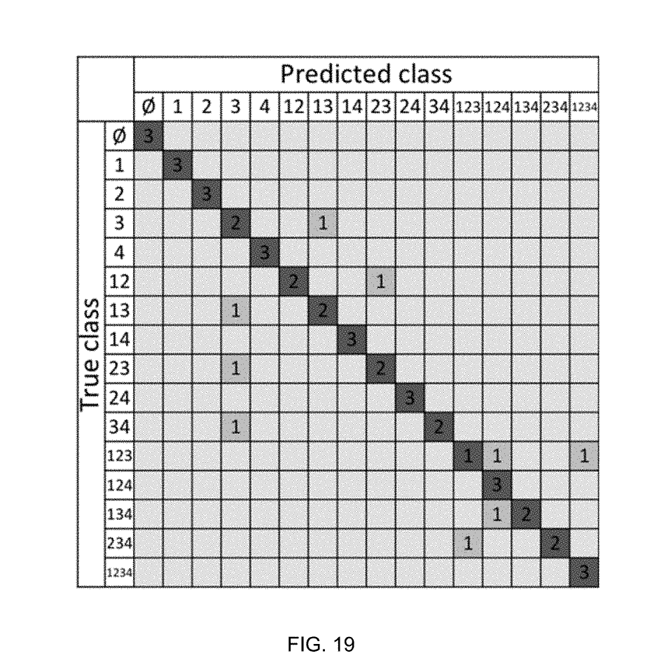

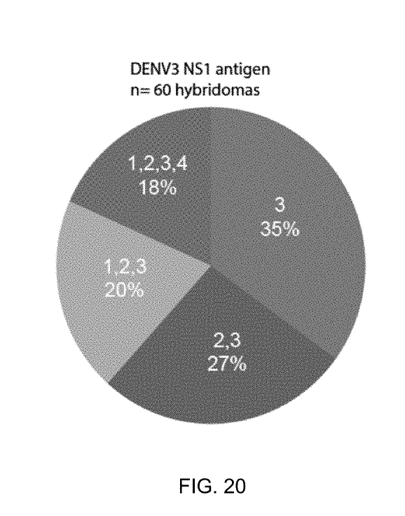

Principal component analysis (PCA) was used to plot the results from six to two dimensions and could successfully separate clusters of the four dengue serotypes and the blank as shown by a confusion matrix (FIG. 6B) with only diagonal values (gray) and no off-diagonals present (white). We also probed the multiplexed assay with mixtures of NS1 and obtained 81% classification accuracy with all the combinations of serotypes (FIG. 19). These results show that it is possible to construct a multiplexed immunoassay entirely out of D3V antibodies that can distinguish all four serotypes of dengue.

Detection of Dengue and Zika

Next, we applied the strategy for multiplexed detection of zika and dengue NS1 using only dengue antibodies. Zika and dengue NS1 have a sequence similarity of ˜54%,²⁰ and cross reactivity of dengue antibodies for zika NS1 has been previously reported (Priyamvada, L., et al. Proceedings of the National Academy of Sciences 113, 7852 (2016). DOI: 10.1073/pnas.1607931113).

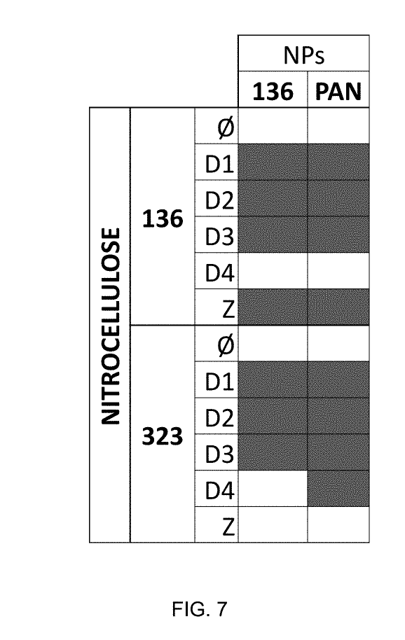

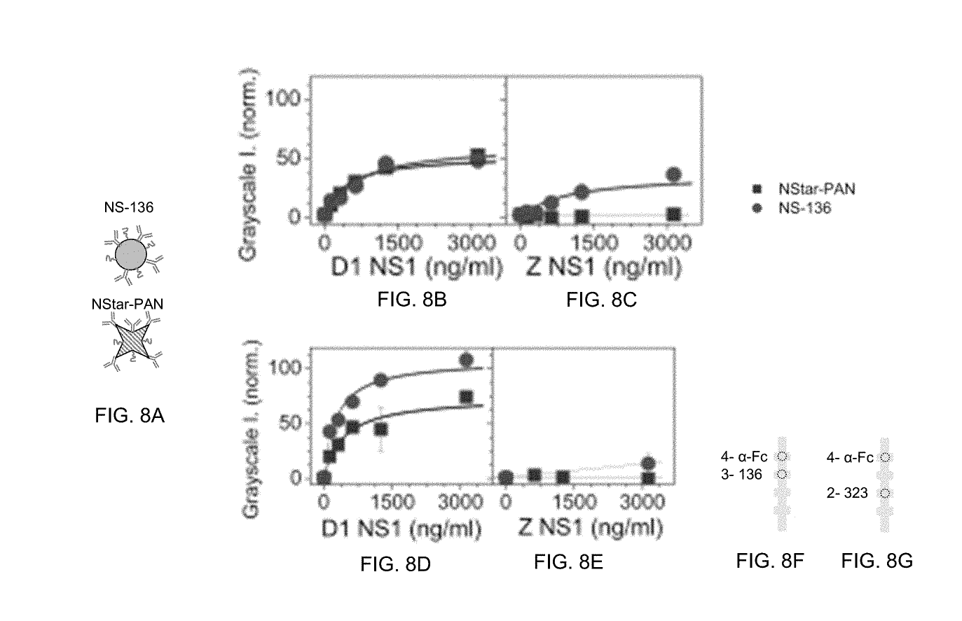

We tested the combinations of dengue antibodies in pairs when run with NS1 from the D1-4V and ZIKV, recording positive tests (gray) and negative ones (white) FIG. 7. We chose a combination of four dengue antibodies to be conjugated to the blue nanostars (GNS-PAN), and as combined they yielded a high intensity when binding to the four serotypes of dengue NS. We conjugated the cross-reactive dengue 136 to the red NPs (GNP-136, FIG. 8A-FIG. 8E) because GNP-136 were able to bind to NS1 from DENV serotypes 1, 2 and 3 as well as ZIKV NS1 (FIG. 7).

We investigated how the immobilized antibodies (136 or 323) could pair with either blue GNS-PAN (squares) or red GNP-136 (circles) when run with D1V or ZIKV NS1 in human serum at different concentrations (0 to 3 ng/ml). For immobilized 136, D1V NS1 was detectable with both GNS-PAN and GNP-136 with similar signal intensities and no signal in the blank, indicating specific detection of D1V NS1 (FIG. 8B, squares and circles, respectively).

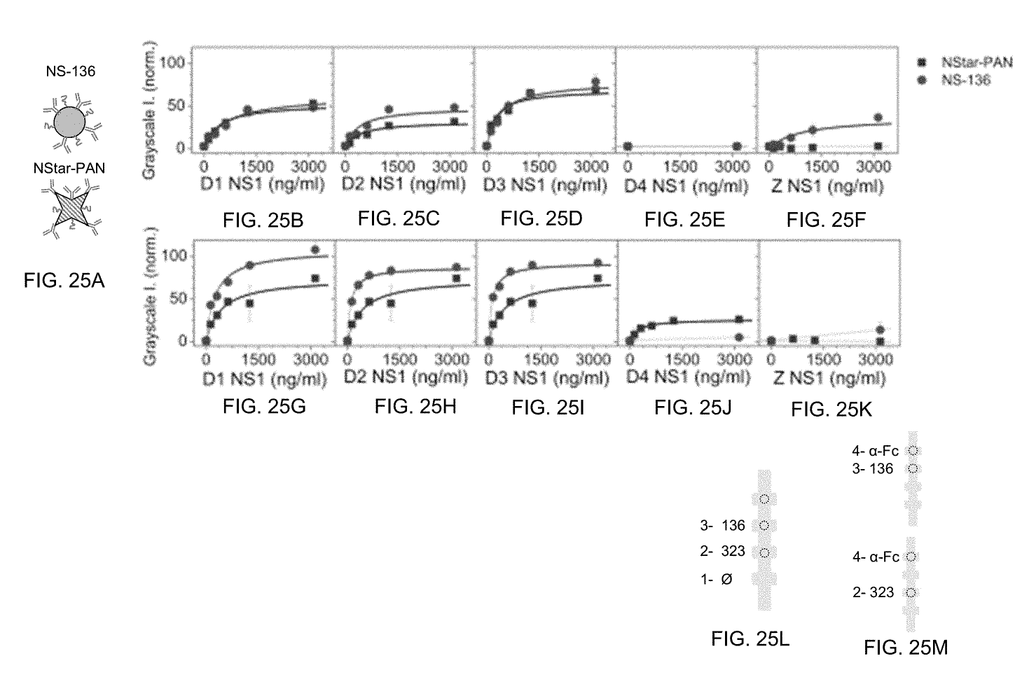

When these pairs were run with ZIKV NS1, only red NS-136 produced a finite signal (circles, FIG. 8C), which was expected from the pairwise analysis (FIG. 7). For immobilized 323, both GNS-PAN and GNP-136 could form sandwiches with D1V NS1 (FIG. 8D), but with different intensities. When running either GNP-136 or GNS-PAN with ZIKV NS1 and immobilized 323 (FIG. 8E), no signal was observed, as expected from the pairwise analysis, which indicated that 323 could detect only dengue NS1. The same analysis was done for the three remaining dengue serotypes D2V-D4V (FIGS. 25B to 25K). We calculated the LODs and K_{DS} of all pairs with ZIKV and all DENV serotypes (FIG. 11), finding LODs between 4-200 ng/ml. This configuration resulted in different RGB intensities depending on whether zika NS1 or dengue NS1 was present.

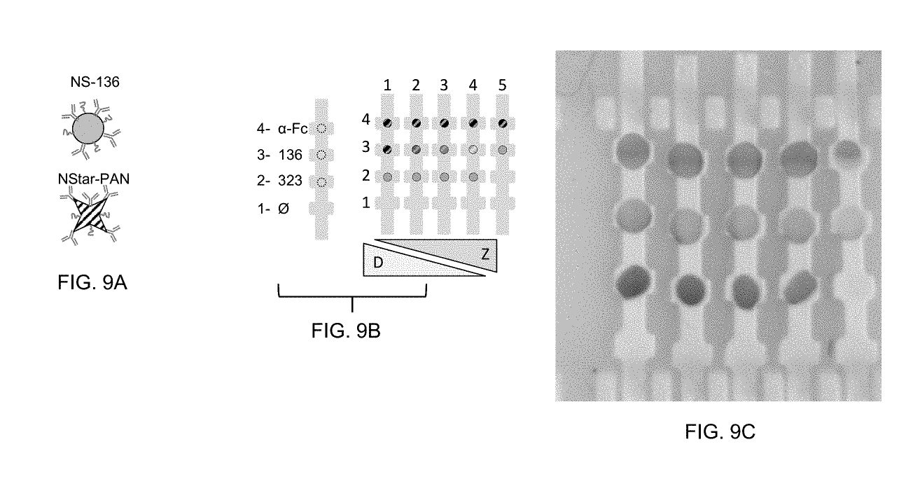

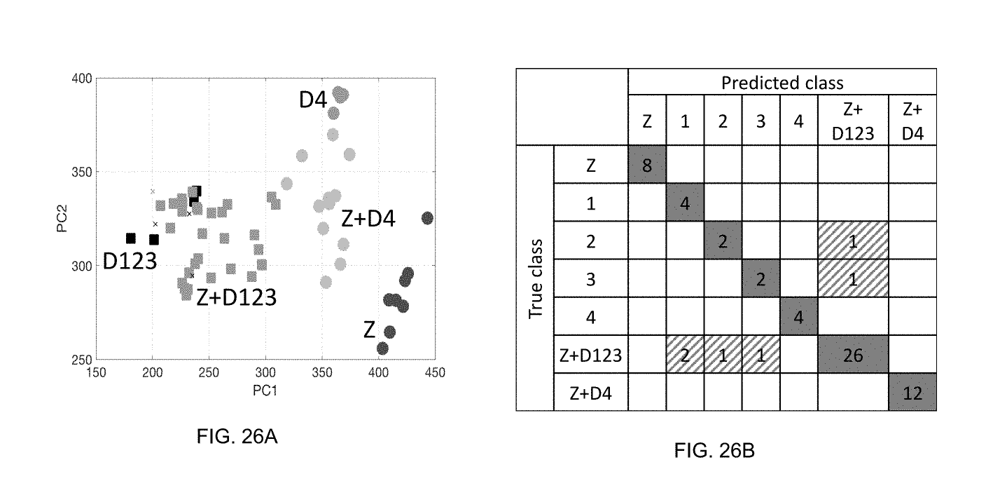

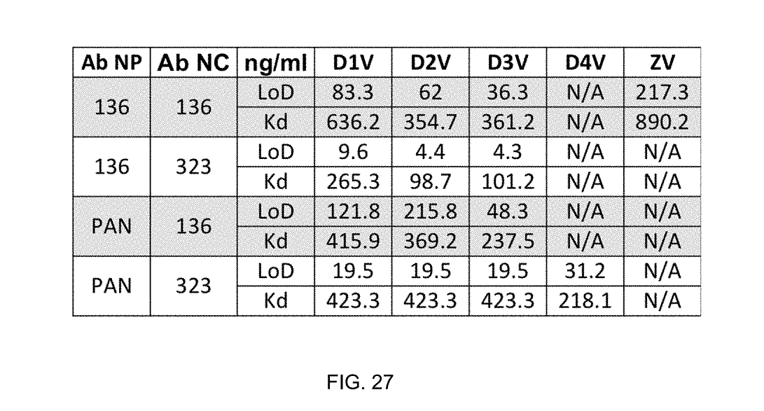

Then, using the selected immobilized Abs and NP-Ab conjugates, we achieved multiplexed detection of zika and dengue by using an arrangement that had immobilized antibodies 323, 136, and anti-Fc at separate areas (FIGS. 9A to 9C). Strips were run with red GNP-136 and blue GNS-PAN for D1V and ZIKV NS1 and mixtures in human serum. When D1V NS1 was present, signal appeared at areas 2 and 3, which was due to sandwich formation of both blue GNS-PAN and GNP-136, as expected. When ZIKV NS1 was present, signal was only at test area 3 due to sandwich formation of NS-136 with immobilized 136. Mixtures of D1V and ZIKV at increasing ratios of D1V to ZIKV showed intermediate intensities at test area 2 where 323 was immobilized (FIG. 9C). Titrations were repeated with the three remaining DENV serotypes. Similar results were obtained except for D4V, resulting in an easier test to interpret (FIGS. 26A-26B).

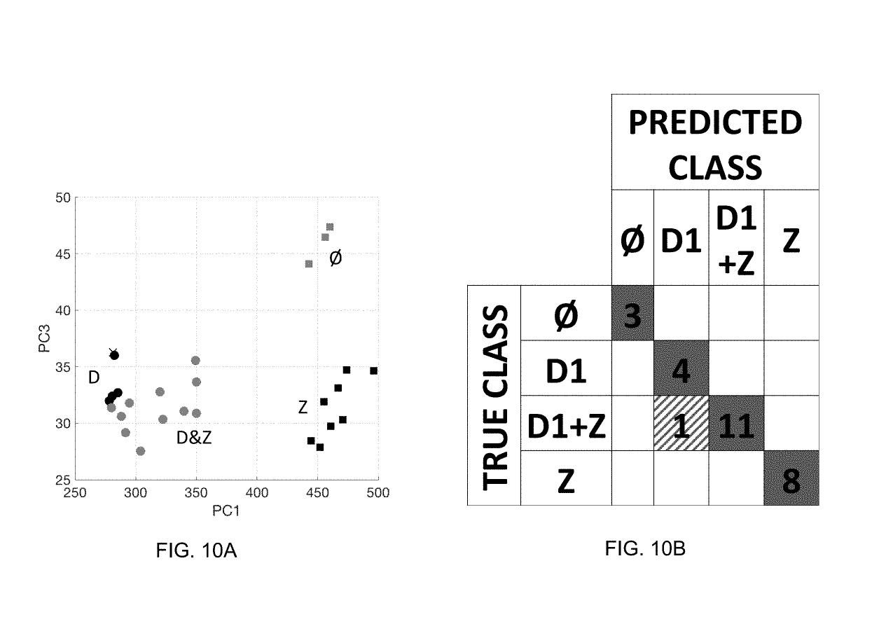

RGB image analysis and subsequent training with LDA showed that we could successfully distinguish D1V from ZV and also the mixtures of D1V& ZIKV with a classifier accuracy of 96%. (on-diagonals, gray, FIG. 10A). Only one test, the 2:1 mixture of D1V: ZIKV NS1, was incorrectly classified (cross-hatched square, FIG. 10B). Assay behavior with dengue-zika NS1 mixtures for all dengue serotypes obtained 92% classification accuracy (FIG. 10B). This shows that it is possible to make a multiplexed ZIKV and DENV immunoassay entirely out of DENV antibodies.

Augmenting a Commercial Dengue Diagnostic for Multiplexed Zika Detection

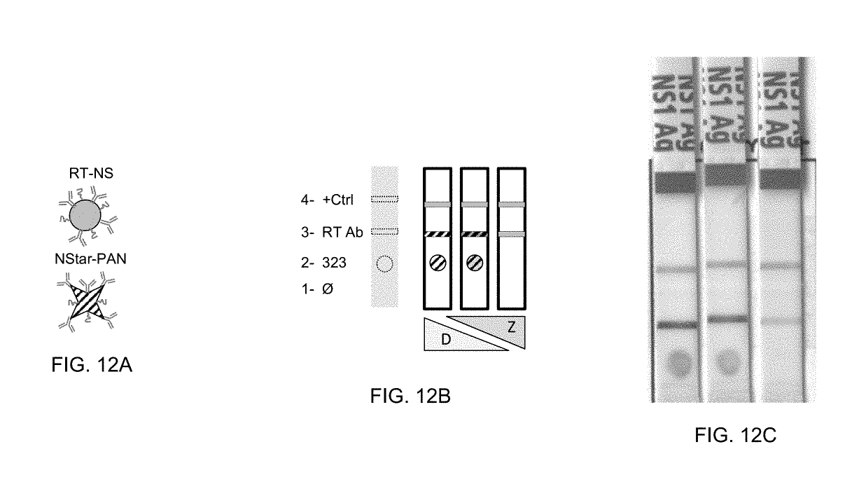

After the zika outbreak, several reports appeared showing that Alere's SD NS1 Ag diagnostic was cross-reactive with dengue and zika (FIG. 11).²¹ Because the diagnostic is cross-reactive with Zika, this indicates that the RT-NS (FIG. 11A and FIG. 11B) and the test line (RT-Ab) form pairs with both DENV and ZIKV NS1. This suggests that augmentation with additional dengue antibodies and NPs of a different color could convert the diagnostic into a multiplexed test for dengue and zika NS1. We thus augmented the diagnostic with another dengue antibody that does not bind to ZIKV NS1 on the strip (323, area 2) and a set on blue GNS (GNS-PAN) to enable differentiation between DENV and ZIKV NS1 (FIG. 11A). Tests were run with NS1 from D1V, ZIKV, and their mixtures. DENV NS1 resulted in a signal at the 323 spot as well as the commercial diagnostic test band RT-Ab (areas 2 and 3, FIG. 12B). The commercial test band was blue, suggesting that GNS-PAN has high affinity for D1V NS1. When ZIKV NS1 was run, a different test pattern resulted, with a red signal at the RT-Ab test band (area 3), but no signal at 323, indicating that commercial RT-NS were binding to ZIKV NS1, but GNS-PAN could not form a sandwich with ZIKV NS1 and 323. For mixtures of D1V and ZIKV NS1, signal appeared at both areas 2 and 3, but the color at the RT-Ab band ranged from blue to purple, while the dot at 323 mostly remained blue (FIG. 12B). Thus, both the resulting test line patterns and color varied depending on whether D1V, ZIKV, or mixtures were present.

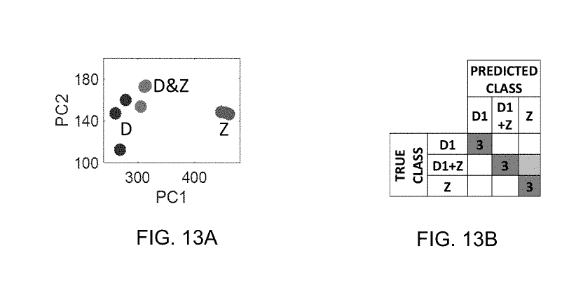

Image analysis of areas 2 and 3 determined that LDA was capable of successfully distinguishing DENV NS1 from ZIKV NS1 and from the mixtures of DENV and ZIKV, having correctly classified the tests (grey squares, FIG. 13B). PCA was used to plot the results from six to two dimensions that could separate the clusters of D1V, ZV and the mixtures of D1V-ZV at different ratios of dengue to zika NS1 (FIG. 13A). Thus, this strategy can be used to augment a commercial diagnostic for multiplexed zika and dengue detection, using only dengue antibodies.

Extension to Filoviruses

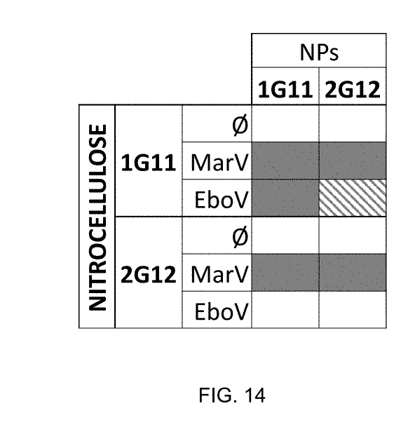

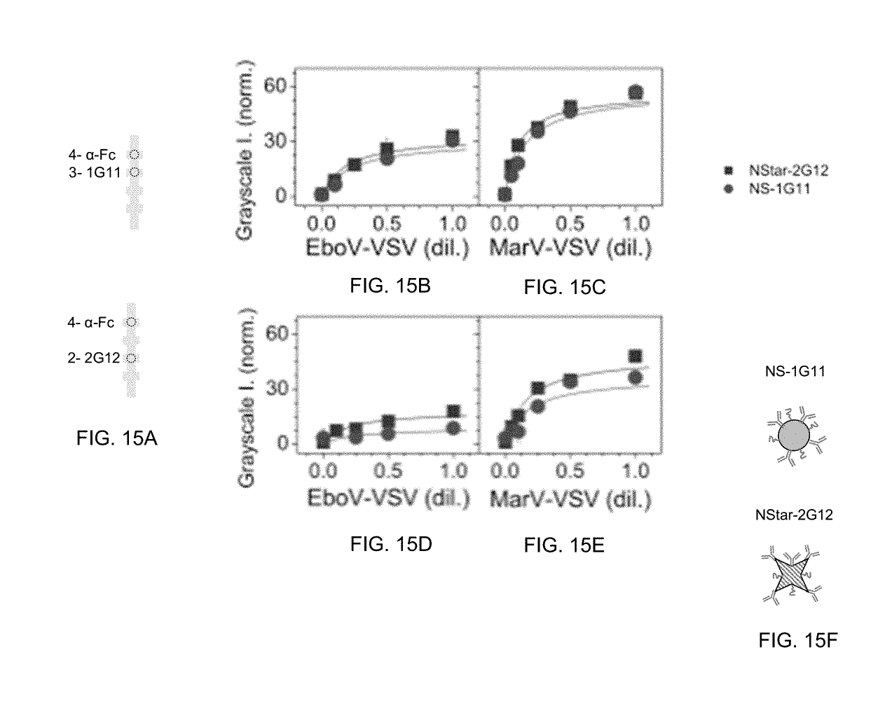

To demonstrate applicability beyond flaviviruses, we investigated Ebola and Marburg, closely related filoviruses that share the secreted biomarker glycoprotein 2 (GP) with a sequence similarity of 31%. We investigated whether it was possible to use Marburg antibodies for multiplexed detection of Marburg and Ebola GP. We tested five antibodies raised from immunizations with Marburg GP. Marburg antibodies were obtained by infecting KH7 cells with a replication-competent vesicular stomatitis virus bearing Ebola GP (EBOV-GP) or Marburg GP (MARV-GP) and used the infection supernatants as the antigen. Antibody pairs that bound to antigen were recorded (FIG. 14, grey), as well as those that did not (white) and those that bound weakly (cross-hatching). 1G11 was capable of pairing with itself with both MARV-GP and EBOV-GP; moreover, antibody 2G12 was cross-reactive as well with 1G11, but not when immobilized on the nitrocellulose (FIG. 14). Based on the antibody behavior, we selected cross-reactive 1G11 to conjugate to red GNPs and 2G12 to blue GNS, and 2G12 for immobilization on test area 2 and 1G11 on area 1 (FIG. 15A-FIG. 15F).

Titration curves were measured for selected antibody pairs with infected supernatant at different dilutions with uninfected media. First, we tested immobilized 1G11 with GNS-2G12 and GNP-1G11. Both NP-Ab could recognize EBOV-GP and resulted in similar intensities (FIG. 15B). For MARV-GP, high signals were observed with both GNS-2G12 and GNP-1G11 (FIG. 15C). Immobilized 2G12 had low signal for both GNS-1G11 and GNP-2G12 when run with EBOV-GP (FIG. 15D), which was expected from the screening (FIG. 14). For MARV-GP, both GNS-2G12 and GNP-1G11 produced signal (FIG. 15E). Again, both immobilized antibodies exhibited different intensities when run with RGB signals when run with EBOV and MARV-GP.

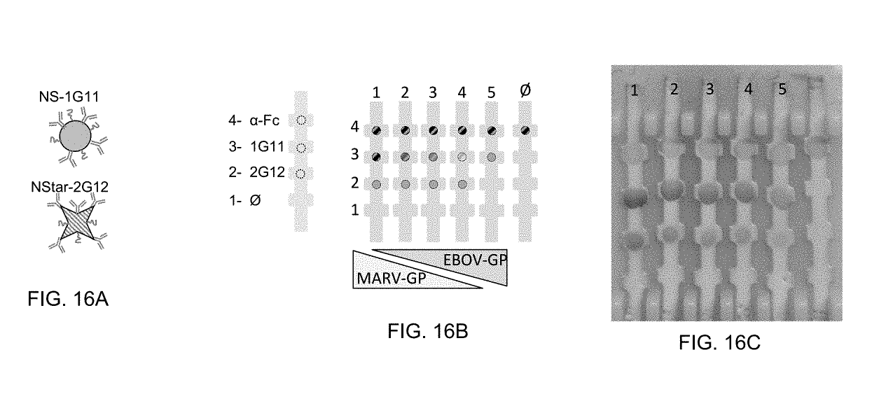

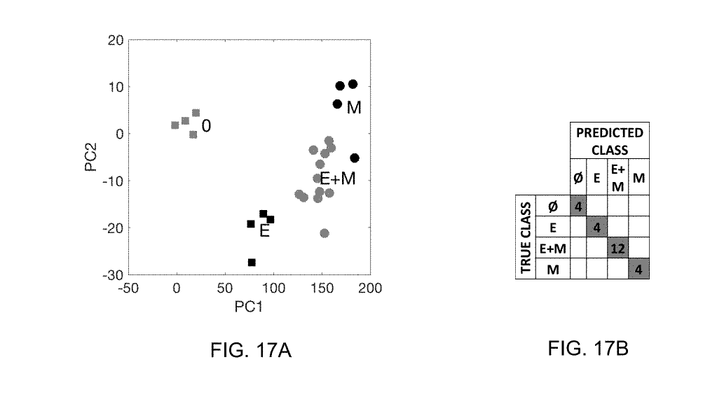

We achieved multiplexed detection by running GNP-1G11 and GNS-2G12 with spatially separated 2G12 and 1G11 (FIG. 16). When EBOV-GP was present, a purple dot formed at area 3. When MARV-GP was present, spots appeared at both areas 2 and 3. EBOV-GP:MARV-GP mixtures yielded spots at areas 2 and 3 with intermediate colors (FIG. 16C). RGB and LDA analysis (FIG. 17A and FIG. 17B) determined that we could distinguish EBOV-GP from MARV-GP and also mixtures with 100% classification accuracy. PCA results could separate the clusters of EBOV-GP, and MARV-GP and the mixtures at different ratios, demonstrating extension to filoviruses.

### Discussion

We demonstrate that immunoassay specificity for a biomarker can be achieved with cross-reactive antibodies if there is a range of cross-reactivity and it is combined with multicolor NPs. Differentiation is enabled because test line intensity is a function of many parameters, including the NP-Ab physical properties, such as Ab coverage on the NP, NP-Ab concentration, and antibody binding affinity, which results in different test line RGB values for the test line depending on the biomarker present. In combination with the spatial arrangement, specific multiplexed detection is possible, which is similar to approaches used in chemical sensor arrays for chemical olfaction.²²

Because the timescale for generating specific antibodies for an emerging outbreak exceeds the initial stages of an outbreak when rapid tests are critically needed, cross-reactivity, normally viewed as a drawback, can actually be exploited. One interesting question is whether this approach is applicable to any set of antibodies. We believe that a higher sequence similarity between the biomarkers facilitates exploiting cross reactivity, and thus do not anticipate that the approach can be used for viruses of a different family (i.e., use flavivirus antibodies for alphavirus detection). We believe that the approach is applicable beyond these two virus families, and an exciting possibility is application for IgG/IgM detection. Furthermore, the approach of analyzing the RGB signal of the test line could be used to detect if an emerging biomarker is present in the sample that is not the one the antibodies are screened for. This approach could also be applied to test lines that use fluorescent or colorimetric beads of different colors for readout.

As with any POC immunoassay, limitations include lower sensitivity and specificity compared to PCR and ELISA. Furthermore, RGB analysis is required, which is not achievable by eye.²³ However, the benefits of having a rapid diagnostic in the critical time period before new specific antibodies are generated could outweigh disadvantages. Inexpensive diagnostics that can deliver results within the hour are increasingly needed for patient treatment and disease management, especially where several diseases with same symptoms co-circulate. This approach could aid rapid response, leveraging of stockpiled antibodies, facilitate rapid turnaround of tests for emerging outbreaks.

### Materials and Methods

Reagents: Au chloride trihydrate was purchased from Sigma-Aldrich (CAS: 16961-25-4). Bis(sulphatophenyl)phenyl-phosphine dehydrate (BPS), was purchased from Aldrich (CAS:308103-66-4). N-(2-Hydroxyethyl) piperazine-N′-(2-ethanesulphonic acid) (HEPES) was purchased from United States Biochemical Company (CAT: 16926). Sodium citrate was from Mallinckrodt Chemicals and 5 kD mPEG was from nanocs. Fluorescent Goat anti-Mouse IgG (H+L) Secondary Antibody, DyLight 650 conjugate was purchased from Pierce.

Synthesis and bioconjugation of gold nanostars: Au NStars with different extinction spectra were synthesized by tuning the Au/HEPES ratio in solution. We mixed 200 μl of 140 mM HEPES (pH 7.4) with 800 μl of 18 MΩ deionized (MilliQ) water, or 500 μl of 140 mM HEPES (pH 7.4) with 500 μl of 18 MΩ deionized (MilliQ) water, for the synthesis of the magenta or blue nanostars, respectively, followed by the addition of 16 μl of 25 mM HAuCl₄.3H₂O and further vortexing. After vortexing, solutions sat undisturbed for 1 h, during which the NStars formed. Afterwards, ˜0.5 mg BPS was added for NStar stabilization, and the solution was vortexed and left undisturbed for 1 h. For antibody conjugation, the NStars were separated from excess reagents by centrifugation at 12000 rcf for 12 min. The resulting NStar pellet was resuspended in 100 μl of 40 mM HEPES at pH 7.7, followed by the addition of 10 μl of 1 mg/ml antibody, vortexed, and further agitated for 45 min, during which time, the antibodies were able to bind to the nanostars. In order to avoid nonspecific binding on the nanostars, 5 μl of 0.1 mM mPEG was added, and the solution was vortexed and further agitated for 20 min, during which time mPEG was able to passivate any bare gold surfaces. Nanoparticles were centrifuged for 12 min at 10000 rcf to separate excess reagents, and then used in the lateral flow tests.

NP Characterization: Optical absorption spectra of the NP were obtained on a Cary 100 UV Vis from Agilent Technologies. Morphology of the NP was characterized with a FEI Tecnai G2 TEM at 120 kV. ImageJ was used to process the images and measure the dimensions of the NP. In addition, a Zetasizer Nano ZS from Malvern Instruments was used to measure the hydrodynamic diameter (DH) and the of the NP. Agarose gel electrophoresis was used to confirm the antibody and mPEG binding on the nanoparticles, in short, 1% agarose gels were prepared, and NPs were loaded by mixing 8 μl of concentrated NPs with 4 μl of 50% glycerol in MilliQ water. Fluorescence spectroscopy was used to quantify the amount of antibodies bound per nanoparticle, by a supernatant-loss method.

Antibodies: Zika and dengue antibody pairs were generated in the lab. Marburg antibodies were a generous donation from FDA.

Bioconjugation of nanoparticles: Innova-coat nanoparticles were conjugated as indicated by the manufacturer. **when more than one antibody is bound, then equal amounts of each are added**

Running the assays: Antibodies were immobilized on nitrocellulose membranes (EDM Millipore HF18002XSS) by manually pipetting 0.3 μl of a 2 mg/ml solution of antibodies onto the nitrocellulose paper, where they automatically were immobilized, and further allowed to dry for at least 30 min. Strips were attached to a wick (GB003 Gel Blot Paper) with adhesive paper (MIBA-010 Backing Card, 0.020″ thickness; DCN Diagnostics, Carlsbad, Calif.). For the positive control area, 0.3 μl of anti-mouse Fc antibody (EDM Millipore AQ127) at 1 mg/ml was spotted on the control line. Strips were placed inside a solution containing: 8 μl of 1% Tween-20 in PBS and 4 μl of 50% sucrose in water, 5 μl of a mixture of NStar and Innova NS and 30 μl of the analyte, typically diluted in filtered human serum, or infected cell supernatants. The tests were run by letting the solution migrate through the strip via capillary action. Once the tests had been dried, images of the finished tests were scanned and quantified with ImageJ.

### REFERENCES

1. Smith, K. F.; Goldberg, M.; Rosenthal, S.; Carlson, L.; Chen, J.; Chen, C.; Ramachandran, S., Global rise in human infectious disease outbreaks. Journal of The Royal Society Interface 2014, 11 (101).
2. Pai, N. P.; Vadnais, C.; Denkinger, C.; Engel, N.; Pai, M., Point-of-Care Testing for Infectious Diseases: Diversity, Complexity, and Barriers in Low- And Middle-Income Countries. PLoS Med 2012, 9 (9), e1001306.
3. Shukla, A. A.; Thömmes, J., Recent advances in large-scale production of monoclonal antibodies and related proteins. 2010, 28 (5), 253-261.
4. Monoclonal Antibody Production; National Research Council (US) Committee on Methods of Producing Monoclonal Antibodies: Washington, D.C., 1999.
5. Gates, B., The Next Epidemic—Lessons from Ebola. New England Journal of Medicine 2015, 372 (15), 1381-1384.
6. Camargo, M. E.; Rebonato, C., Cross-Reactivity in Fluorescence Tests for Trypanosoma and Leishmania Antibodies: A Simple Inhibition Procedure to Ensure Specific Results. The American Journal of Tropical Medicine and Hygiene 1969, 18 (4), 500-505.
7. Moulin, E.; Selby, K.; Cherpillod, P.; Kaiser, L.; Boillat-Blanco, N., Simultaneous outbreaks of dengue, chikungunya and Zika virus infections: diagnosis challenge in a returning traveller with nonspecific febrile illness. 2016, 11, 6-7.
8. Knowlton, K.; Solomon, G.; Rotkin-Ellman, M., Mosquito-borne dengue fever threat spreading in the Americas. In Issue paper, National Resource Defense Council: 2009.
9. Borgherini, G.; Poubeau, P.; Jossaume, A.; Gouix, A.; Cotte, L.; Michault, A.; Arvin-Berod, C.; Paganin, F., Persistent arthralgia associated with chikungunya virus: A study of 88 adult patients on Reunion Island. Clinical Infectious Diseases 2008, 47 (4), 469-475.
10. Bhatt, S.; Gething, P. W.; Brady, O. J.; Messina, J. P.; Farlow, A. W.; Moyes, C. L.; Drake, J. M.; Brownstein, J. S.; Hoen, A. G.; Sankoh, O.; Myers, M. F.; George, D. B.; Jaenisch, T.; Wint, G. R. W.; Simmons, C. P.; Scott, T. W.; Farrar, J. J.; Hay, S. I.; Inst, M., The Global Distribution and Burden of Dengue. Influence of Global Environmental Change on Infectious Disease Dynamics: Workshop Summary 2014, 297-310.
11. Yen, C.-W.; de Puig, H.; Tam, J. O.; Gomez-Marquez, J.; Bosch, I.; Hamad-Schifferli, K.; Gehrke, L., Multicolored silver nanoparticles for multiplexed disease diagnostics: distinguishing dengue, yellow fever, and Ebola viruses. Lab on a Chip 2015, 15 (7), 1638-41.
12. Watanabe, S.; Tan, K. H.; Rathore, A. P. S.; Rozen-Gagnon, K.; Shuai, W.; Ruedl, C.; Vasudevan, S. G., The Magnitude of Dengue Virus NS1 Protein Secretion Is Strain Dependent and Does Not Correlate with Severe Pathologies in the Mouse Infection Model. Journal of Virology 2012, 86 (10), 5508-5514.
13. Xie, J.; Lee, J. Y.; Wang, D. I. C., Seedless, Surfactantless, High-Yield Synthesis of Branched Gold Nanocrystals in HEPES Buffer Solution. Chem. Mater. 2007, 19 (11), 2823-2830.
14. de Puig, H.; Tam, J. O.; Yen, C.-W.; Gehrke, L.; Hamad-Schifferli, K., The extinction coefficient of gold nanostars. Journal of Physical Chemistry C 2015, 119 (30), 17408-17415.
15. Badu-Tawiah, A. K.; Lathwal, S.; Kaastrup, K.; Al-Sayah, M.; Christodouleas, D. C.; Smith, B. S.; Whitesides, G. M.; Sikes, H. D., Polymerization-based signal amplification for paper-based immunoassays. Lab on a Chip 2015, 15 (3), 655-659.
16. de Puig, H.; Cane-Camps, M.; Bosch, I.; Hamad-Schifferli, K., Effect of the protein corona on antibody-antigen binding in nanoparticle sandwich immunoassays. Bioconjugate Chemistry 2017, 28 (1), 230-238.
17. Tam, J. O.; Puig, H. d.; Yen, C.-w.; Bosch, I.; Gomez-Marquez, J.; Clavet, C.; Hamad-Schifferli, K.; Gehrke, L., A Comparison of Nanoparticle-Antibody Conjugation Strategies in Sandwich Immunoassays. Journal of Immunoassay and Immunochemistry 2017, 1-23.
18. Young, P. R.; Hilditch, P. A.; Bletchly, C.; Halloran, W., An antigen capture enzyme-linked immunosorbent assay reveals high levels of the dengue virus protein NS1 in the sera of infected patients. Journal of Clinical Microbiology 2000, 38 (3), 1053-1057.
19. Abramoff, M. D.; Magelhaes, P. J.; Ram, S. J., Image Processing with ImageJ. Biophotonics International 2004, 11 (7), 36-42.
20. Song, H.; Qi, J.; Haywood, J.; Shi, Y.; Gao, G. F., Zika virus NS1 structure reveals diversity of electrostatic surfaces among flaviviruses. Nature Structural and Molecular Biology 2016, advance online publication.
21. Rakow, N. A.; Suslick, K. S., A colorimetric sensor array for odour visualization. Nature 2000, 406 (6797), 710-713.
22. Gyurech, D.; Schilling, J.; Schmidt-Chanasit, J.; Cassinotti, P.; Kaeppeli, F.; Dobec, M., False positive dengue NS1 antigen test in a traveller with an acute Zika virus infection imported into Switzerland. Swiss Medical Weekly 2016, 146, w14296.
23. Lee, S.; Mehta, S.; Erickson, D., Two-Color Lateral Flow Assay for Multiplex Detection of Causative Agents Behind Acute Febrile Illnesses. Analytical Chemistry 2016, 88 (17), 8359-8363.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims. It should also be understood that the preferred embodiments described herein are not mutually exclusive and that features from the various preferred embodiments may be combined in whole or in part in accordance with the invention.

1. A multiplexed immunoassay method comprising:
a) contacting the biological sample with one or more detection antibodies wherein at least one detection antibody of the one or more detection antibodies is cross reactive for an antigenic site on a target infectious disease biomarker protein to which the antibody was raised and is also capable of binding to an antigenic site on a different target protein, wherein at least one detection antibody of the one or more detection antibodies is capable of forming a complex with at least one target protein in the sample, wherein all of the one or more detection antibodies are labelled with a unique colorimetric label comprising a unique spectral emission;
b) contacting the biological sample of step (a) with a porous matrix comprising one or more capture antibodies immobilized thereon in a capture-detection area of the porous matrix, wherein at least one capture antibody of the one or more capture antibodies is specific for an antigenic site on the target infectious disease biomarker protein to which the antibody was raised and optionally wherein at least one capture antibody of the one or more capture antibodies is capable of binding an antigenic site on a different target protein, and wherein the complexes formed in step (a) migrate through the porous matrix and contact the immobilized capture antibodies in the capture-detection area of the porous matrix;
(c) detecting the differential color and intensity pattern of one or more unique spectral emissions in the capture-detection area of the porous matrix; and
(d) identifying one or more species of infectious disease biomarker protein and different target protein, present in the sample based on the differential color and intensity pattern of the unique spectral emissions in the capture-detection area of the porous matrix.

2. The method of claim 1, wherein the porous matrix comprises nitrocellulose.

3. The method of claim 1, wherein the unique colorimetric label is selected from: gold nanoparticles, colored latex beads, carbon nanoparticles, selenium nanoparticles, silver nanoparticles quantum dots, up converting phosphors, organic fluorophores.

4. The method of claim 1 wherein a colorimetric sensor is used in the detection of the differential color and intensity pattern of the spectral emissions of step (c).

5. The method of claim 4, wherein colorimetric sensor detects red-green-blue (RGB) values of the color and intensity pattern of the spectral emissions of step (c).

6. The method of claim 5, wherein the colorimetric sensor is a mobile phone comprising an RGB color analysis application installed thereon.

7. The method of claim 1, wherein the target protein in the sample is from a virus.

8. The method of claim 7, wherein the target protein from a virus is a non-structural protein-1 (NS1) or a glycoprotein (GP).

9. The method of claim 8, wherein the virus is Dengue virus, Zika virus, Yellow fever virus, West Nile virus, Ebola virus, or Marburg virus.

10. The method of claim 1, wherein the biological sample is derived from a human patient.

11. The method of claim 1, wherein the colorimetric label is a gold nanoparticle.

12. The method of claim 1, wherein the porous matrix is nitrocellulose and is formatted as a dipstick lateral flow assay (LFA).

13. The method of claim 1, wherein the identifying step (d) comprises comparing the differential color and intensity pattern in the capture-detection area of the porous matrix to the differential color and intensity pattern of a pre-screening assay comprising the detection antibodies and the capture antibodies in the presence of a known amount of target protein.

14. The method of claim 1, wherein there are at least two capture antibodies present in the capture-detection area and the at least two capture antibodies are spaced in the capture-detection area such that their spectral emissions do not interfere with each other.

15. A method of identifying one or more antibody pairs that are cross reactive for a target infectious disease biomarker protein and a different target protein comprising:
(a) immobilizing each species of antibody to be tested in the capture-detection area of a porous matrix thereby providing a capture antibody;
(b) labeling each species of antibody to be tested with a unique colorimetric label comprising a unique spectral emission thereby providing a detection antibody;
(c) adding the labeled detection antibodies of step (b) to a biological sample comprising a predetermined amount of at least one known infectious disease biomarker protein and optionally, a predetermined amount of at least one different target protein for a sufficient time to allow the labeled detection antibodies to form a complex with one or more target proteins;
(d) contacting the biological sample of step (c) with the porous matrix of step (a);
(e) detecting the differential color and intensity pattern of the spectral emissions in the capture-detection area of the porous matrix; and
(f) identifying the antibody pairs comprising a capture antibody and a detection antibody that show specificity for only one target protein or that show cross reactivity with more than one target protein based on the differential color and intensity pattern of the spectral emissions in the capture-detection area of the porous matrix.

16. The method of claim 15, wherein in step (c) the known infectious disease biomarker protein and the optional different target protein comprises a protein derived from a virus of the same family.

17. The method of claim 16, wherein the viral family from which the target protein is derived is the flavivirus family or the filovirus family.

18. The method of claim 17, wherein target protein is NS1 derived from a virus of the flavivirus family.

19. The method of claim 18, wherein the flavivirus is dengue virus, Yellow Fever virus, Powassan or zika virus.

20. The method of claim 17, wherein the target protein is GP derived from a virus of the filovirus family.

21. The method of claim 20, wherein the filovirus is Ebola virus or Marburg virus.

22. A kit for the detection of zika virus, dengue virus or both in a biological sample comprising:
(a) a commercially available lateral flow immunoassay for detecting dengue virus using anti-dengue antibodies specific to the NS1 protein of one or more serotypes of dengue virus;
(b) an anti-dengue antibody that is cross reactive with the NS1 protein of both dengue virus and zika virus and that is labelled with a unique colorimetric label comprising a unique spectral emission thereby providing a cross-reactive detection antibody; and
(c) an anti-dengue antibody capture antibody known to pair with the cross reactive anti-dengue antibody of (b).

23. The method of claim 1, wherein the different target protein of step (a) is a previously unidentified infectious disease protein wherein antibodies have not previously be raised to the different target protein.

24. The method of claim 15, wherein the different target protein of step (c) is a previously unidentified infectious disease protein wherein antibodies have not previously be raised to the different target protein.
